(19) United States
(12) Patent Application Publication
(10) Pub. No.: US 2026/0345640 A1
(43) Pub. Date: Nov. 5, 2026

(54) COMPOSITIONS AND METHODS FOR TETRACYCLINE ANTIBIOTIC RESCUE

(71) Applicants: Washington University, St. Louis, MO (US); The United States of America, represented by the Secretary, Department of Health and Human Services, Bethesda, MD (US)

(72) Inventors: Ruihao Li, St. Louis, MO (US); Yao-Peng Xue, St. Louis, MO (US); Wai Kwan Tang, Bethesda, MD (US); Niraj Harish Tolia, Bethesda, MD (US); Gautam Dantas, St. Louis, MO (US); Timothy A. Wencewicz, St. Louis, MO (US)

(21) Appl. No.: 19/665,676

(22) Filed: May 1, 2026

Related U.S. Application Data
(60) Provisional application No. 63/798,793, filed on May 2, 2025.

(51) Int. Cl. A61K 31/65; A61P 31/04

(52) U.S. Cl. CPC A61K 31/65; A61P 31/04

(57) ABSTRACT

Tetracycline destructases (TDases) oxidize tetracycline antibiotics into inactive products via reactions with the flavin adenine dinucleotide (FAD) cofactor. This disclosure relates to compositions and methods involving covalent modification of the FAD cofactor using anhydrotetracycline molecules having warheads capable of reacting with FAD. The formation of a covalent adduct between FAD and the anhydrotetracycline molecules is disclosed as a new class of inhibitors against TDases. The inhibitors comprise anhydrotetracycline derivatives modified at the C9 position. Biochemical and whole cell assays demonstrate that propargylamine and cyclopropylamine warheads are effective against Type I and II TDases. Such inhibitors can be used in therapies to rescue antibiotic activity of tetracycline antibiotics. Pharmaceutical compositions and therapeutic methods are disclosed.

### CROSS REFERENCE TO RELATED APPLICATIONS

This application claims priority under 35 U.S.C. § 119(e) to provisional patent application U.S. Ser. No. 63/798,793, filed May 2, 2025. The provisional patent application is hereby incorporated by reference in its entirety herein, including without limitation: the specification, claims, and abstract, as well as any figures, tables, appendices, or drawings thereof.

### STATEMENT REGARDING FEDERALLY SPONSORED RESEARCH OR DEVELOPMENT

This invention was made with government support under AI123394 awarded by the National Institutes of Health. The government has certain rights in the invention.

### NAMES OF THE PARTIES TO A JOINT RESEARCH AGREEMENT

The subject matter disclosed was developed and the claimed invention was made by, or on behalf of, one or more parties to a joint research agreement that was in effect on or before the effective filing date of the claimed invention; the claimed invention was made as a result of activities undertaken within the scope of the joint research agreement; and the parties of the joint research agreement are Washington University in St. Louis and the National Institutes of Health.

### TECHNICAL FIELD

The present disclosure relates generally to compositions for rescuing tetracycline antibiotic activity and methods of use.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Tetracycline (TC) antibiotics have been an important class of antibacterial agents since the first discovery of aureomycin (chlortetracycline) in the late 1940s. As a family of type-II polyketides, TC antibiotics are constructed with a fused system of four linear rings. TC antibiotics demonstrate impressive antibacterial properties against a wide range of pathogens, including both Gram-positive and Gram-negative bacteria. The antibiotic activity of TCs is achieved by interfering with bacterial protein biosynthesis through binding to the 16S rRNA of the 30S bacterial ribosomal subunit and preventing the binding and accommodation of amino-acyl tRNAs. Extensive research and development efforts have been devoted to optimizing TC antibiotics through semi synthesis and total synthesis to keep pace with resistance caused by efflux and ribosome protection protein mechanisms. The development of second-generation TCs, such as doxycycline and minocycline, improved the pharmacokinetic properties and increased the efficacy against resistant bacterial species expressing TC efflux pumps. Third- and fourth-generations of TC antibiotics, such as tigecycline, omadacycline, and eravacycline have been developed to overcome resistance via efflux pumps and ribosomal protection proteins.

Thus, there exists a need in the art for a clinically useful TC antibiotic or drug which address enzymatic inactivation as a new clinical resistance mechanism.

### SUMMARY

Next-generation tetracycline antibiotics are threatened by enzymatic inactivation as emerging resistance mechanism. The relevant enzymes, tetracycline destructases (TDases), are structural homologues of class A flavin monooxygenase (FMO) that oxidize tetracycline antibiotics, leading to various inactive degradation products. Small molecule inhibitors of antibiotic-inactivating enzymes are critical clinical therapeutics used to manage bacterial resistance with combination therapy. While reversible TDase inhibitors have been reported, covalent inhibitors were developed that are better aligned with clinically effective covalent β-lactamase inhibitors. Here, the design, chemical synthesis, and biochemical characterization are reported of the first covalent irreversible inhibitors of TDases based on C9-derivatives of anhydrotetracycline (aTC). The reactive warheads were installed via a one-step Mannich reaction linking either an N-(1-methyl)cyclopropylamine or N-propargylamine group to the C9-position of the aTC D-ring via an amino methylene linkage. Two nonspecific FMO inhibitors, N-(1-methyl)cyclopropylbenzylamine (101) and N-methyl-N-benzyl-propargylamine (102) were synthesized as mechanistic probes to distinguish reactivity with the essential FAD cofactor in TDases via one- or two-electron transfer pathways, respectively. The compounds were evaluated as potential inhibitors of representative TDases from the two major classes, Type 1 (TetX6 and TetX7) and Type 2 (Tet50). The aTC-based compounds 103, 104, and 105 inhibited both Type 1 and Type 2 TDases with notable differences in potency and inhibition mechanism. The inhibition of Type 1 TDases was more potent but reversible with no time dependence. The inhibition of Type 2 TDases was time-dependent and irreversible even after exhaustive dialysis, consistent with a covalent mechanism of inhibition. Molecular modeling of the inhibitors supports unique inhibitor binding modes for Type 1 and Type 2 TDases that are consistent with the observed differences in the inhibition modes. Blue light irradiation of the Type 2 TDase enhanced this reactivity. Treatment of Tet50 with probe molecules 101 and 102 under blue light exposure enabled the identification of covalent FAD adducts via mass spectrometry that are consistent with the expected one- and two-electron transfer reaction modes of the cyclopropylamine and propargylamine warheads with the FAD cofactor. At concentrations as low as 2 μg/mL, the aTC-based covalent inhibitors 103, 104, and 105 recovered tetracycline activity against E. coli overexpressing TDases. The findings suggest that the inhibition of TDases through covalent trapping of the FAD cofactor is a viable strategy for overcoming TDase-mediated antibiotic resistance.

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

A preferred embodiment is a composition for treating tetracycline resistance comprising: a tetracycline compound, wherein the tetracycline compound comprises tetracycline, a tetracycline derivative, a tetracycline analog, a pharmaceutically acceptable salt of the foregoing, and/or mixtures thereof; wherein the tetracycline compound has a warhead capable of forming a covalent bond with a flavin adenine dinucleotide.

A preferred embodiment is a method of treating a bacterial infection in a subject, the method comprising: administering a therapeutically effective amount of a composition, wherein the composition comprises; tetracycline compound, wherein the tetracycline compound comprises tetracycline, a tetracycline derivative, a tetracycline analog, a pharmaceutically acceptable salt of the foregoing, and/or mixtures thereof; wherein the tetracycline compound has a warhead capable of forming a covalent bond with a flavin adenine dinucleotide.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments. Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows a mechanistic basis of the function of MAO and TDase enzymes, cyclopropylamine (101) and propargylamine (102) reactive warheads, and incorporation of the reactive warheads into a aTC scaffold.

FIG. 2A shows x-ray crystal structures of aTC bound to Tet50 on the left and TetX6 on the right.

FIG. 2B shows a proposed mechanism for covalent adduct formation between FAD and a propargylamine via a two-electron pathway.

FIG. 2C shows a proposed mechanism for covalent adduct formation between FAD and a cyclopropylamine via a one-electron pathway.

FIG. 3A shows a structure of N-(1-methyl)cyclopropylbenzylamine (101) and the corresponding photoinduced FAD covalent adduct, optical absorbance spectra measured at 1-hour intervals over an 18-hour period from Tet50 samples treated with (101) without external light in bottom left graph, and absorbance spectra measured at 1-minute intervals over a 10-minute period from Tet50 samples treated with (101) with blue light in bottom right graph.

FIG. 3B shows a structure of N-methyl-N-benzyl-propargylamine (102) and the corresponding photoinduced FAD covalent adduct, optical absorbance spectra measured at 1-hour intervals over an 18-hour period from Tet50 samples treated with (102) without external light in bottom left graph, and absorbance spectra measured at 1-minute intervals over a 10-minute period from Tet50 samples treated with (102) with blue light in bottom right graph.

FIG. 4A shows a structure of the Tet50 inhibitor aTC and x-ray crystal structure of aTC-bound Tet50.

FIG. 4B shows a structure of a docked aTC cyclopropylamine 103 overlapping with aTC in the Tet50 active site.

FIG. 4C shows a structure of a docked aTC propargylamine 104 overlapping with aTC in the Tet50 active site.

FIG. 5 shows syntheses of C9-cyclopropylamine and C9-propargylamine aTC derivatives 103, 104, and 105 via one-step Mannich reactions.

FIG. 6A shows half-maximal inhibitory concentration IC₅₀ plots (top) and apparent IC₅₀ values versus time (bottom), top plots showing preincubation times with aTC (first from left), compound 103 (second from left), compound 104 (third from left), and compound 105 (fourth from left) for Tet50 under dark incubation without exposure to external light.

FIG. 6B shows IC₅₀ plots (top) and apparent IC₅₀ values versus time (bottom), top plots showing preincubation times with aTC (first from left), compound 103 (second from left), compound 104 (third from left), and compound 105 (fourth from left) for Tet50 under blue light.

FIG. 6C shows IC₅₀ plots (top) and apparent IC₅₀ values versus time (bottom), top plots showing preincubation times with aTC (first from left), compound 103 (second from left), compound 104 (third from left), and compound 105 (fourth from left) for TetX7 under dark incubation without exposure to external light.

FIG. 7A shows a plot of inhibited Tet50 activity under dark incubation conditions prior- and post-dialysis.

FIG. 7B shows a plot of inhibited Tet50 activity under blue light incubation conditions prior- and post-dialysis.

FIG. 7C shows inhibited TetX7 activity under dark incubation conditions prior- and post-dialysis.

FIG. 8 shows synthesis of N-(1-methyl)cyclopropylbenzylamine (101) (top) and synthesis of N-methyl-N-benzyl-propargylamine (102) (bottom).

FIG. 9 shows SDS-PAGE analysis of N-6-His tagged TDases and para-hydroxybenzoate dydroxylase (PHBH) enzymes after elution from Ni-NTA resin, dialysis, and concentration in storage buffer using Precision Plus Protein™ Dual Xltra Prestained Protein Standards #1610397 to measure enzyme mass.

FIG. 10A shows a series of half-maximal inhibitory concentration (IC₅₀) curves for inhibition of Tet50 degradation of tetracycline.

FIG. 10B shows a series of IC₅₀ curves for inhibition of TetX7 degradation of tetracycline.

FIG. 10C shows a series of IC₅₀ curves for inhibition of TetX6 degradation of tetracycline.

FIG. 11 shows a plot comparing the photostability of the following the class A flavin monooxygenase enzymes: Tet50, TetX7, TetX6, and PHBH.

FIG. 12A shows a plot of UV-Vis spectral changes measured for the Tet50 control protected from external light with measurements at 1-hour intervals for 18 hours.

FIG. 12B shows a plot of UV-Vis spectral changes measured for the Tet50 control under blue light with measurements at 1-minute intervals for 10 minutes.

FIG. 13A shows the structure of Tet50-101 adduct (left), high resolution mass spectrometry analysis of Tet50-101 adduct (top right), and liquid chromatography-mass spectrometry of Tet50-101 adduct (bottom right).

FIG. 13B shows the structure of Tet50-102 adduct (left), high resolution mass spectrometry analysis of Tet50-102 adduct (top right), and liquid chromatography-mass spectrometry of Tet50-102 adduct (bottom right).

FIG. 14 shows a one-electron mechanism for the formation of an FAD covalent adduct derived from compound 102.

FIG. 15A shows a plot of spectral changes measured from PHBH samples treated with compound 101 and exposed to blue light.

FIG. 15B shows a plot of spectral changes measured from PHBH samples treated with compound 102 and exposed to blue light.

FIG. 15C shows a plot of spectral changes measured from PHBH samples treated with compound 101 and kept in the dark.

FIG. 15D shows a plot of spectral changes measured from PHBH samples treated with compound 102 and kept in the dark.

FIG. 16 shows an overlay of the docked and co-crystallized aTC in Tet50.

FIG. 17A shows docking of compound 103 against an aTC:TetX6 cocrystal structure.

FIG. 17B shows docking of compound 104 against an aTC:TetX6 cocrystal structure.

FIG. 18A shows a time-dependent IC₅₀ plot for Tet50 under various pre-incubation times with compound 101.

FIG. 18B shows a time-dependent IC₅₀ plot for Tet50 under various pre-incubation times with compound 102.

FIG. 18C shows a time-dependent IC₅₀ plot for PHBH under various pre-incubation times with compound 101.

FIG. 18D shows a time-dependent IC₅₀ plot for PHBH under various pre-incubation times with compound 102.

FIG. 19A shows a time-dependent IC₅₀ plot (top) for PHBH under various pre-incubation times with aTC without light exposure and apparent IC₅₀ values versus time (bottom).

FIG. 19B shows a time-dependent IC₅₀ plot (top) for PHBH under various pre-incubation times with compound 103 without light exposure and apparent IC₅₀ values versus time (bottom).

FIG. 19C shows a time-dependent IC₅₀ plot (top) for PHBH under various pre-incubation times with compound 104 without light exposure and apparent IC₅₀ values versus time (bottom).

FIG. 20 shows ¹H NMR plot of compound 101.

FIG. 21 shows ¹³C NMR plot of compound 101.

FIG. 22 shows ¹H NMR plot of compound 102.

FIG. 23 shows ¹H NMR plot of compound 103.

FIG. 24 shows ¹H NMR plot of compound 104.

FIG. 25 shows ¹H NMR plot of compound 105.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

From the foregoing, it can be seen that the present disclosure accomplishes at least all of the stated objectives.

It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

As used herein, the term “exemplary” refers to an example, an instance, or an illustration, and does not indicate a most preferred embodiment unless otherwise stated.

The term “substantially” refers to a great or significant extent. “Substantially” can thus refer to a plurality, majority, and/or a supermajority of said quantifiable variables, given proper context.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4. This applies regardless of the breadth of the range.

The term “or” is synonymous with “and/or” and means any one member or combination of members of a particular list.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The term “about,” as used herein, refers to variation in the numerical quantity that can occur, for example, through typical measuring techniques and equipment, with respect to any quantifiable variable, including, but not limited to, concentration, conformation, distance, dosage, energy, free energy, length, mass, pH, temperature, time, absorbance, radiation, enzyme activity, and molecular weight. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Alkenyl groups or alkenes are straight chain, branched, or cyclic alkyl groups having 2 to about 30 carbon atoms, and further including at least one double bond. In some embodiments, alkenyl groups have from 2 to about 20 carbon, or typically, from 2 to 10 carbon atoms. Alkenyl groups may be substituted or unsubstituted. Alkenyl groups may be substituted similarly to alkyl groups.

As used herein, the terms “alkylene”, “cycloalkylene”, “alkynylene”, and “alkenylene”, alone or as part of another substituent, refer to a divalent radical derived from an alkyl, cycloalkyl, or alkenyl group, respectively, as exemplified by —CH2CH2CH2-. For alkylene, cycloalkylene, alkynylene, and alkenylene groups, no orientation of the linking group is implied.

As used herein, “aryl” or “aromatic” groups are cyclic aromatic hydrocarbons that do not contain heteroatoms. Aryl groups include monocyclic, bicyclic, and polycyclic ring systems. Thus, aryl groups include, but are not limited to, phenyl, azulenyl, heptalenyl, biphenylenyl, indacenyl, florenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, in others from 6 to 12 or 6-10 carbon atoms in the ring portions of the groups. The phrase “aryl groups” includes groups containing fused rings, such as fused aromatic-aliphatic ring systems. Aryl groups may be substituted or unsubstituted.

In the present disclosure, an “effective amount” or “therapeutically effective amount” of a compound or of a composition of the present disclosure is that amount of such compound and/or composition that is sufficient to effect beneficial or desired results as described herein. In terms of treatment of a mammal, e.g., a human patient, an “effective amount” is an amount sufficient to treat, reduce, manage, palliate, ameliorate, or stabilize a condition as compared to the absence of the compound or composition.

The terms “include” and “including” when used in reference to a list of materials refer to but are not limited to the materials so listed.

As used herein, “pharmaceutically acceptable” refers to compounds, materials, compositions, and/or dosage forms which are suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. A “pharmaceutically acceptable excipient” includes any and all carriers, solvents, growth media, dispersion media, coatings, adjuvants, fillers, buffers, stabilizers, lubricants, stabilizing agents, diluents, preservatives, inactivating agents, antimicrobial, antibacterial and antifungal agents, isotonic agents, adsorption delaying agents, and the like. Such ingredients include those that are safe and appropriate for use in medical or pharmaceutical applications.

The term “subject” as used herein refers to any living being that would benefit from the compositions and methods described herein. For example, the subject may be an animal, including a human, avian, bovine, canine, equine, feline, hircine, lupine, murine, ovine, and porcine animal. Subjects may also be domesticated animals such as cats, dogs, rabbits, guinea pigs, ferrets, hamsters, mice, gerbils, horses, cows, goats, sheep, donkeys, pigs, and the like. In certain embodiments, the subject is a human.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

### Compositions

One aspect of the present disclosure encompasses a composition for treating a bacterial infection in a subject. In an embodiment, the composition may comprise a compound that interacts with, binds to, and/or inhibits a flavoenzyme.

In an embodiment, the composition comprises a tetracycline compound. In a further embodiment, the tetracycline compound comprises a tetracycline, a tetracycline derivate, a tetracycline analog, a pharmaceutical acceptable salt of the foregoing, and/or mixtures thereof.

In a further embodiment, the tetracycline compound has a warhead capable of forming a covalent bond with a flavin adenine dinucleotide.

In a further embodiment, the composition comprises a covalent flavoenzyme inhibitor compound that is a tetracycline compound as detailed above.

In a further embodiment, the composition further comprises a tetracycline antibiotic.

### Flavoenzymes

Tetracycline destructases (TDases) are members of the flavoenzyme group. Specifically, tetracycline destructases are members of the class A flavin monooxygenase (FMO) enzyme family. FMOs are single component flavoprotein oxidases that utilize a dynamic noncovalent flavin adenine dinucleotide (FAD) as a redox cofactor. The FAD, known in the family of enzymes as the ‘wavin flavin’, can occupy two distinct conformations referred to as ‘FAD-IN’ and ‘FAD-OUT’. The FAD cofactor rests in the TDase active site in its oxidized form. Substrate binding triggers the two-electron reduction of FAD by NAD(P)H, presumably in the ‘FAD-OUT’ conformation. The resulting reduced form FADH₂ presumably moves to the ‘FAD-IN’ conformation where it is subsequently reoxidized by O₂ to form a reactive C4a-peroxyflavin species. Substrate oxidation commonly takes place via oxygen transfer (hydroxylation) or oxygen insertion (Baeyer-Villiger type) resulting in a C4a-hydroxyflavin that dehydrates to the original resting FAD oxidized state.

Two major families of TDases, Type 1 and Type 2, have been reported. Type 1 TDases are more frequently encountered in clinically isolated strains, whereas the Type 2 enzymes are more common in environmental microbes. Type 2 TDases differ from Type 1 TDases in the presence of an extra C-terminal ‘gatekeeper’ helix that allows for closing of the active site during catalysis. This structural variation between subtypes leads to other distinguishing properties including substrate scope, substrate binding mode, product outcome, susceptibility to inhibitors, and photo-stability. The increased occurrence of TDases in clinical pathogens and ability to inactivate all generations of TC drugs creates an urgent need for effective inhibitors.

In an embodiment, the composition comprises a compound that interacts with and/or inhibits a flavoenzyme.

In some embodiments, the flavoenzyme is a member of the class A flavin monooxygenase (FMO) enzyme group.

In a further embodiment, the flavoenzyme is a tetracycline destructase. Further, the tetracycline destructase can be a Type I or Type II tetracycline destructase. Further, the tetracycline destructase is a Type II or Type II tetracycline destructase selected from the group consisting of Tet47, Tet48, Tet49, Tet50, Tet51, Tet52, Tet54, Tet55, Tet56, TetX, and combinations thereof

### Covalent Flavoenzyme Inhibitor Compounds

An example of treating infections caused by bacterial pathogens expressing antibiotic inactivating enzymes is the use of combination drug therapy. For example, β-Lactam antibiotics such as amoxicillin are commonly coadministered with a β-lactamase inhibitor such as clavulanic acid. This type of combination therapy could be applied to TC antibiotics against pathogens expressing TDases as a primary resistance mechanism. It was previously reported that anhydrotetracycline (aTC), a TC biosynthetic precursor and autoinducer, is a competitive inhibitor of Type 1 and Type 2 TDases. The structural basis for the inhibition of Type 1 and Type 2 TDases by aTC was determined. This revealed two distinct inhibitor binding modes for each type. The inhibitor binding mode directs the design of various aTC-based inhibitors for different types of TDases. The Type 2 TDase Tet50 (PDB: 5TUF) binds aTC with the D-ring pointing toward the active site cavity in the ‘FAD-OUT’ conformation. The Type 1 TDase TetX6 (PDB: 8ER0) flips the aTC 180° along the relative y-plane to position the A-ring toward the inside of the active site cavity with D-ring exposed to solvent at the entrance of the active site in the ‘FAD-IN’ conformation. Using structure-based drug design, a series of bivalent TDase inhibitors were developed. The inhibitors were based on C9-benzamide or C10-benzoate derivatives of aTC that bind competitively and reversibly to the TC and NADPH sites. While these bivalent inhibitors showed promising synergy with TC antibiotics, the potency and efficacy of the inhibitors could be improved via a covalent inhibition mechanism.

In an embodiment, the composition comprises a compound that interacts with and/or inhibits a flavoenzyme.

Covalent irreversible enzyme inhibitors offer several potential therapeutic advantages over reversible inhibitors such as enhanced potency, longer target dwell time, and reduced dosage frequency. As a result, the development and application of covalent inhibitors has gained popularity in clinical applications including the inhibition of antibiotic inactivating enzymes. For example, clavulanic acid, sulbactam, avibactam, and vaborbactam are all FDA-approved covalent inhibitors of β-lactamases that are used in combination with β-lactam antibiotics. β-Lactamases contribute to antibiotic resistance by hydrolyzing the thermodynamically labile β-lactam ring that is conserved among this antibiotic class. All clinically useful β-lactamase inhibitors are covalent inhibitors that react with the conserved active serine residue present in all Class A, C, and D β-lactamases. The clinical success of β-lactamase inhibitors suggests that covalent inhibitors could play an essential role in combating antibiotic resistance for a wide range of antibiotic inactivating enzymes including TDases.

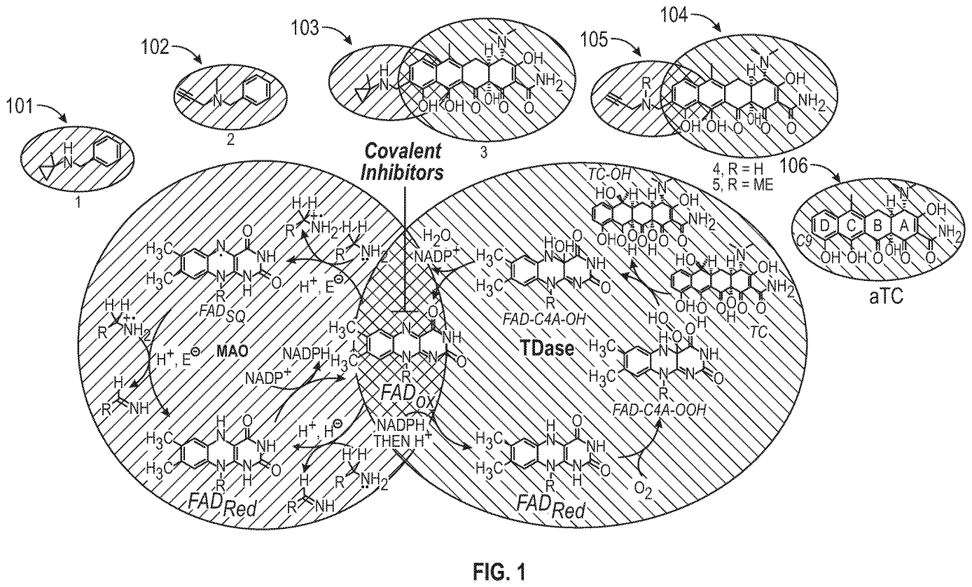

As class A FMOs, TDases present two general strategies for covalent inhibition: 1) covalent tagging of amino acid side chains, or 2) covalent reactions with the FAD cofactor. The covalent adduct formation between the inhibitor and the FAD cofactor could result in a tightly bound inhibition complex. The known TDase inhibitor aTC can be functionalized with two types of reactive warheads, a propargylamine or cyclopropylamine group, which are known to undergo redox-triggered covalent tagging of the FAD cofactor in monoamine oxidases (MAOs). MAOs oxidize amines to imines through direct oxidation by FAD, which is capable of mediating redox reactions via two-electron and/or one-electron transfer pathways (FIG. 1). Propargylamines exploit the two-electron pathway, whereas cyclopropylamines exploit the one-electron pathway. Both pathways lead to the formation of electrophilic intermediates that can react with the nucleophilic N5 of the reduced FAD isoalloxazine ring leading to covalent adduct formation via N5-alkylation.

During a two-electron process, a reactive FAD anion intermediate, [FADH]⁻, is formed. Propargylamine inhibitors of MAOs were found to form covalent adducts with the FAD cofactor presumably resulting from a two-electron transfer mechanism involving the [FADH]⁻ anion. Propargylamine derivatives have been utilized since the 1950s for the treatment of neuropsychiatric and age-related neurodegenerative disorders via the covalent inhibition of MAOs. Extensive studies have elucidated the mechanism of covalent adduct formation between propargylamine derivatives and MAOs. The first step involves a hydride transfer from the propargylamine to N5 of the oxidized FAD. Subsequently, the resulting iminium cation is trapped by the nucleophilic N5 of the reduced [FADH], forming a substituted 1,3-diaminoallene through a conjugate addition reaction. The resulting intermediate then undergoes a water-mediated 1,3-prototropic rearrangement leading to a highly conjugated covalent adduct.

During a one-electron process, a reactive FAD semiquinone intermediate, [FADH]^{⋅}, is formed. Cyclopropylamine inhibitors of pig liver mitochondrial MAOs were found to form covalent adducts with the FAD cofactor presumably resulting from a one-electron transfer mechanism involving the [FADH]^{⋅} semiquinone. The initial step of this mechanism involves single electron transfer (SET) from the cyclopropylamine nitrogen lone pair to the oxidized FAD, generating the amine radical cation and the [FADH]^{⋅} semiquinone after protonation. The amine radical cation activates the adjacent C—C bond of the cyclopropyl ring toward α-cleavage followed by radical recombination with the [FADH]^{⋅} semiquinone resulting in FAD N5-alkylation.

Given that both MAOs and TDases contain an oxidized FAD cofactor in the resting state, the modes of reactivity observed for MAOs with propargylamines and cyclopropylamines can be extended to TDases to provide covalent inhibitors. Both one- and two-electron modes of reactivity can be enhanced via photoactivation of FAD. Under visible light irradiation, oxidized FAD can be excited to the singlet state (1F1*) and subsequently undergo a rapid intersystem crossing (ISC) to the highly reactive triplet state (3F1*), which is calculated to be a better oxidizing agent than ground-state FAD. Therefore, a new type of TDase inhibitor was designed merging the scaffolds of the known TDase inhibitor aTC and known MAO inhibitors cyclopropylamine 101 and propargylamine 102 resulting in the synthesis of C9-cyclopropylamine-aTC analog 103 and C9-propargylamine-aTC analogs 104 and 105. The reactivity of compounds 101, 102, 103, 104, and 105 toward Type 1 and Type 2 TDases was investigated using time-dependent kinetic assays and exhaustive dialysis under both ‘dark’ and ‘light’ conditions. The ability of compounds 103, 104, and 105 to synergistically rescue TC activity against E. coli expressing TDases was demonstrated.

In some embodiments, the flavoenzyme is a member of the class A flavin monooxygenase (FMO) enzyme group.

In a further embodiment, the flavoenzyme is a tetracycline destructase. Further, the tetracycline destructase can be a Type 1 or Type II tetracycline destructase.

In some embodiments, the composition comprises a compound that inhibits a flavoenzyme via a covalent inhibition mechanism. Class A flavin monooxygenase enzymes utilize flavin adenine dinucleotide (FAD) as a redox cofactor. In an embodiment, the compound forms a covalent adduct as a result of a covalent reaction with FAD.

Exemplary compounds that interact with and/or inhibit a flavoenzyme include, without limit, anhydrotetracycline, chlortetracycline, oxytetracycline, demeclocycline, a derivative, an analog, pharmaceutically acceptable salts of the foregoing, and/or combinations thereof. In an embodiment, the compound that interacts with and/or inhibits a flavoenzyme is an anhydrotetracycline, a chlortetracycline, an oxytetracycline, a demeclocycline, and combinations thereof. In preferred embodiment, the compound that interacts with and/or inhibits a flavoenzyme is an anhydrotetracycline or derivative thereof. In a further embodiment, the compound that inhibits a flavoenzyme is a C9-derivative of anhydrotetracycline.

In an embodiment, the compound has a warhead. As used herein, the term “warhead” or “warhead group” refers to a functional group present on a compound of the present invention wherein that functional group is capable of covalently binding to an enzyme or enzyme cofactor, resulting in irreversible covalent inhibition. In an embodiment, the warhead is positioned at the C9 position of a tetracycline. In an embodiment, the warhead is a cyclopropylamine or a propargylamine. In a further embodiment, the warhead is positioned at the C9 position of an anhydrotetracycline.

Pharmaceutically acceptable salts of a compound that interacts with and/or inhibits a flavoenzyme include, without limit, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

### Tetracycline Compounds

In an embodiment, the composition comprises a tetracycline compound. Tetracycline compounds include tetracycline, tetracycline derivatives, tetracycline analogs, pharmaceutically acceptable salts of the forefoing, and mixtures thereof. Tetracyclines are broad-spectrum antibiotics belonging to a subclass of polyketides having an octahydrotetracene-2-carboxamide skeleton. Exemplary tetracycline compounds include, without limit, tetracycline, chlortetracycline, demeclocycline, doxycycline, epi-tetracycline, epi-anhydrotetracycline, anhydrotetracycline, lymecycline, meclocycline, metacycline, methacycline, minocycline, oxytetracyline, tigecycline, and combinations thereof, including derivatives, analogs, and/or pharmaceutically acceptable salts of the foregoing and mixtures thereof.

In a preferred embodiment, the tetracycline compound comprises anhydrotetracycline, a derivative of anhydrotetracycline, an analog of anhydrotetracycline, a pharmaceutically acceptable salt of the foregoing, and/or mixtures thereof.

Pharmaceutically acceptable salts of a tetracycline compound include, without limit, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

### Tetracycline Antibiotics

In an embodiment, the composition further comprises a tetracycline antibiotic. The tetracycline antibiotic can be used in a combination therapy as outlined above. In an embodiment, the composition comprises a tetracycline compound and a tetracycline antibiotic.

Exemplary tetracycline antibiotics include, without limit, tetracycline, chlortetracycline, demeclocycline, doxycycline, epi-tetracycline, epi-anhydrotetracycline, anhydrotetracycline, lymecycline, meclocycline, metacycline, methacycline, minocycline, oxytetracyline, tigecycline, and combinations thereof, including derivatives, analogs, and/or pharmaceutically acceptable salts of the foregoing and mixtures thereof.

### Pharmaceutical Compositions

The present disclosure also provides pharmaceutical compositions. The pharmaceutical composition comprises a tetracycline compound, a tetracycline antibiotic, a small molecule, a polynucleotide, a polypeptide, a vector or isolated cell, as an active ingredient, at least one pharmaceutically acceptable excipient, and combinations thereof. In a preferred embodiment, the pharmaceutical composition comprises a tetracycline compound as detailed above. In an embodiment, the pharmaceutical composition further comprises a tetracycline antibiotic.

The pharmaceutically acceptable excipient may be a diluent, a binder, a filler, a buffering agent, a pH modifying agent, a disintegrant, a dispersant, a preservative, a lubricant, taste-masking agent, a flavoring agent, a coloring agent, and/or combinations thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, trehalose, and or combinations thereof. Non-limiting examples of suitable abrasively brittle diluents include dibasic calcium phosphate (anhydrous or dihydrate), calcium phosphate tribasic, calcium carbonate, magnesium carbonate, and/or combinations thereof.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and/or combinations thereof.

In another embodiment, the excipient may be a filler. Suitable fillers include, but are not limited to, carbohydrates, inorganic compounds, polyvinylpyrrolidone, and/or combinations thereof. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, sorbitol, and/or combinations thereof.

In still another embodiment, the excipient may be a buffering agent. Representative examples of suitable buffering agents include, but are not limited to, phosphates, carbonates, citrates, tris buffers, buffered saline salts (e.g., Tris buffered saline, or phosphate buffered saline), and/or combinations thereof.

In various embodiments, the excipient may be a pH modifier. By way of non-limiting example, the pH modifying agent may be sodium carbonate, sodium bicarbonate, sodium citrate, citric acid, phosphoric acid, and/or mixtures thereof.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, tragacanth, and/or combinations thereof. Non-limiting examples of suitable effervescent disintegrants include sodium bicarbonate in combination with citric acid, sodium bicarbonate in combination with tartaric acid, and/or combinations thereof.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, microcrystalline cellulose, and/or combinations thereof.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate, and/or combinations thereof. Non-limiting examples of suitable preservatives include chelators such as EDTA and/or EGTA. Non-limiting examples of suitable preservatives include antimicrobials such as parabens, chlorobutanol, phenol, and/or combinations thereof.

In a further embodiment, the excipient may be a lubricant. Non-limiting examples of suitable lubricants include minerals such as talc and/or silica, fats such as vegetable stearin, magnesium stearate, or stearic acid, and/or combinations thereof.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and/or combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and/or combinations thereof.

In still a further embodiment, the excipient may be a coloring agent. Suitable color additives include, but are not limited to, food, drug and cosmetic colors (FD&C), drug and cosmetic colors (D&C), external drug and cosmetic colors (Ext. D&C), and/or combinations thereof.

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

### Dosage Forms

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, vehicles, and/or combinations thereof as desired. Topical administration may also involve the use of transdermal administration such as transdermal patches or iontophoresis devices. The term parenteral as used herein includes subcutaneous, intravenous, intramuscular, or intrasternal injection, infusion techniques, and/or combinations thereof. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, caplets, pills, powders, pellets, granules, and/or combinations thereof. In such solid dosage forms, the active ingredient is ordinarily combined with one or more pharmaceutically acceptable excipients, examples of which are detailed above. Oral preparations may also be administered as aqueous suspensions, elixirs, syrups, and/or combinations thereof. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, other synthetic solvents, and/or combinations thereof; an antibacterial and/or antifungal agent such as benzyl alcohol, methyl paraben, chlorobutanol, phenol, thimerosal, and/or combinations thereof; an antioxidant such as ascorbic acid and/or sodium bisulfite; a chelating agent such as etheylenediaminetetraacetic acid; a buffer such as acetate, citrate, or phosphate; and/or an agent for the adjustment of tonicity such as sodium chloride, dextrose, a polyalcohol such as mannitol and/or sorbitol, and/or combinations thereof. The pH of the aqueous solution may be adjusted with acids or bases such as hydrochloric acid or sodium hydroxide. Oil-based solutions or suspensions may further comprise sesame, peanut, olive oil, mineral oil, and/or combinations thereof.

### Methods of Treatment

An additional aspect of the present disclosure encompasses methods for treating a bacterial infection in a subject. The method comprises administering to a subject a therapeutically effective amount of a compound that interacts with, binds to, and/or inhibits a flavoenzyme.

In an embodiment, the compound is a tetracycline compound as described above. In addition, the method can further comprise administering to a subject a therapeutically effective amount of a tetracycline antibiotic. In an embodiment, the tetracycline antibiotic exhibits antibiotic activity against a bacterial infection. In a further embodiment, the tetracycline compound rescues antibiotic activity of the tetracycline antibiotic.

In an embodiment, the tetracycline compound forms a covalent adduct with a flavin adenine dinucleotide.

In an embodiment, the covalent adduct binds to and/or inhibits a flavoenzyme

### Bacterial Infection

In an embodiment, the bacterial infection may be tetracycline resistant.

In an embodiment, the bacterium may encode a tetracycline-inactivating enzyme or tetracycline destructase. In a further, embodiment, the bacterium may be mutated to overexpress a tetracycline-inactivating enzyme or tetracycline destructase as compared to the wild-type bacterium.

### Inhibition

In an embodiment, the compounds described above may interact with a flavoenzyme. In an embodiment, interaction with a flavoenzyme involves binding and/or inhibition. In an embodiment, the compounds bind to the active site of a flavoenzyme or tetracycline destructase. Exemplary members of the tetracycline destructase family of enzymes include, without limit, Tet47, Tet48, Tet49, Tet50, Tet51, Tet52, Tet53, Tet54, Tet55, Tet56, and TetX.

In a preferred embodiment, the compounds described in above bind to and inhibit a tetracycline destructase. Inhibition involves blocking the enzyme from oxidizing a tetracycline into an inactive form.

In an embodiment, the compounds described above bind to and inhibit a flavoenzyme or tetracycline destructase at a different site.

### Administration

In certain aspects, a pharmacologically effective amount of a composition as described in above may be administered to a subject. Administration is performed using standard effective techniques, known to those skilled in the art.

A composition as described above may be formulated and administered to a subject by several different means. For instance, a composition may generally be administered parenterally, intraperitoneally, intravascularly, or intrapulmonarily in dosage unit formulations containing conventional nontoxic pharmaceutically acceptable adjuvants, carriers, excipients, vehicles, and/or combinations thereof as desired. The term parenteral as used herein includes subcutaneous, intravenous, intramuscular, intrathecal, or intrasternal injection, infusion techniques, and/or combinations thereof. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Such compositions can be administered orally, parenterally, by inhalation spray, rectally, intradermally, transdermally, and/or topically in dosage unit formulations containing conventional nontoxic pharmaceutically acceptable carriers, adjuvants, vehicles, and/or combinations thereof as desired. Topical administration may also involve the use of transdermal administration such as transdermal patches or iontophoresis devices. The term parenteral as used herein includes subcutaneous, intravenous, intramuscular, or intrasternal injection, infusion techniques, and/or combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, other synthetic solvents, and/or combinations thereof; an antibacterial and/or antifungal agent such as benzyl alcohol, methyl paraben, chlorobutanol, phenol, thimerosal, and/or combinations thereof; an antioxidant such as ascorbic acid and/or sodium bisulfite; a chelating agent such as etheylenediaminetetraacetic acid; a buffer such as acetate, citrate, phosphate, and/or combinations thereof; and/or an agent for the adjustment of tonicity such as sodium chloride, dextrose, a polyalcohol such as mannitol and/or sorbitol, and/or combinations thereof. The pH of the aqueous solution may be adjusted with acids or bases such as hydrochloric acid or sodium hydroxide. Oil-based solutions or suspensions may further comprise sesame, peanut, olive oil, mineral oil, and/or combinations thereof.

For topical (e.g., transdermal or transmucosal) administration, penetrants appropriate to the barrier to be permeated are generally included in the preparation. Transmucosal administration may be accomplished through the use of nasal sprays, aerosol sprays, tablets, suppositories, and/or combinations thereof, and transdermal administration may be via ointments, salves, gels, patches, creams, and/or combinations thereof as generally known in the art.

The amount of agent that is administered to the subject can and will vary depending upon the type of agent, the subject, and the particular mode of administration. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference.

### EXAMPLES

Embodiments of the disclosed methods are further defined in the following nonlimiting Examples. These Examples, while indicating certain embodiments of the disclosed methods, are given by way of illustration only and should not be considered as limiting in any way. From the above discussion and these Examples, one skilled in the art can ascertain the essential characteristics of this disclosure, and without departing from the spirit and scope thereof, can make various changes and modifications of the embodiments of the disclosed methods to adapt it to various usages and conditions. Thus, various modifications of the embodiments of the disclosed methods, in addition to those shown and described herein, will be apparent to those skilled in the art from the foregoing description. Such modifications are also intended to fall within the scope of the appended claims.

### Example 1

### General Materials and Methods

Organic solvents, including deuterated NMR solvents and reagent-grade chemicals used in the preparation or analysis of synthetic compounds, were purchased from Millipore-Sigma (St. Louis, MO) unless otherwise stated and were used without further purification. aTC (HCl salt) was purchased from Chemodex (United Kingdom). 1-Methylcyclopropanamine (HCl salt) was purchased from AmBeed (Arlington Hts, IL). FAD (disodium salt hydrate) was purchased from Chem-Impex (Wood Dale, IL). All in vitro assays were prepared open to air in nondegassed buffer solutions. NMR spectra were obtained on a Varian Unity-Plus 300 MHz, Varian Unity-Inova 500 MHz, or Agilent PremiumCompact+ 600 MHz spectrometer in 5 mm type 1, class A borosilicate glass NMR tubes (Wilman LabGlass part no. 535-PP-8). All free induction decay files (FIDs) were processed using Mestrenova software (version 12.0.4). Chemical shifts (8) were reported in parts per million (ppm) and referenced to residual nondeuterated solvent. Coupling constants (J) were reported in hertz (Hz). All in vitro TDase and FAD reactions were monitored by measuring optical absorbance (A400) on an Agilent Cary 50 UV-visible spectrophotometer using polystyrene cuvettes or a Tecan Infinite M Plex microplate reader using Costar 96-well transparent polystyrene plates. All enzyme concentrations were determined by measuring optical absorbance (A280) on a Thermo Fisher NanoDrop 2000c spectrophotometer. Low resolution LC-MS was performed using an Agilent 6130 single quadrupole instrument with G1313 autosampler, G1315 diode array detector, and 1200 series solvent module and separated using a Phenomenex Gemini C18 column, 50×2 mm (5 μm) with guard column cassette and a linear gradient of 0% acetonitrile and 0.1% formic acid to 95% acetonitrile and 0.1% formic acid over 25 min at a flow rate of 0.5 mL/min before analysis by electrospray ionization (ESI+). Synthetic compounds were purified by preparative HPLC using an HP1050 system and a reverse phase Luna 10 mm C18 (2) 100 Å column (250 mm×21.2 mm) from Phenomenex fitted with a guard column of the same matrix (15 mm×21.2 mm). HPLC solvents were 0.1% formic acid in H2O (A) and 0.1% formic acid in ACN (B), with a gradient formed from 0% B to 95% B over 20 min at a flow rate of 9 mL/min. LC-MS and HPLC data were processed using ChemStation software version B.04.02 SP1. SDS-PAGE analysis was carried out using Bio-Rad Any kD precast polyacrylamide gels with staining by Coomassie brilliant blue and comparison to a Bio-Rad Precision Plus Protein Dual Xtra prestained protein standard ladder. The molecular formulas and structures of all synthetic compounds were confirmed through HRMS and multidimensional NMR analyses, respectively. The purity of the compounds was confirmed to be >95% by analytical LCMS prior to all biological testing.

### Example 2

### Cloning, Expression, and Purification of TDases

All genes encoding FMOs used in this study (tetX6—QHN11884.1; tetX7—AMP54443.1; tet50—AKQ05894.1; pobA (PHBH)—P00438) were cloned into the BamHI/NdeI (tetX6, tetX7, and tet50) or NdeI/HindII (pobA) restriction sites of a pET28b(+) vector (Novagen) with a 6-His tag at the N-terminus. The cloned construct was transformed into BL21-Star (DE3) competent cells (Life Technologies). The pobA gene encoding for PHBH was ordered from GenScript and was codon optimized for expression in E. coli. Cells were cultured at 37 °C in lysogeny broth (LB) containing 0.03 mg/mL kanamycin (final concentration); once the culture reached an OD₆₀₀ of ~0.6, the cells were cooled to 4 °C. Protein expression was induced by the addition of 1 mM IPTG (final concentration), and cells were grown at 15 °C for 12-15 h. To harvest protein, the induced cells were pelleted by centrifugation at 5000 rpm for 15 min (4 °C) and resuspended in lysis buffer (50 mM K₂HPO₄, 500 mM NaCl, 20 mM imidazole, 10% glycerol, 5 mM BME, pH 8.0) containing SIGMAFAST protease inhibitor (Millipore-Sigma) and cooled to 4 °C. Cell suspensions were transferred to falcon tubes, flash frozen in liquid nitrogen and stored at −80 °C. Frozen cell suspensions were thawed and lysed using a Qsonica ultrasonicator, and the resultant lysate was clarified via ultracentrifugation at 45,000 rpm for 35 min at 4 °C. The clarified supernatant was transferred to a fritted column containing washed and equilibrated Ni-NTA resin and incubated for 30-45 min with gentle rocking. The resin was then washed with lysis buffer (2×40 mL), and the protein was eluted from the resin with elution buffer (4×10 mL elution, 50 mM K₂HPO₄, 500 mM NaCl, 5 mM BME, 300 mM imidazole, 10% glycerol, pH 8.0). Fractions containing the desired proteins (as judged by SDS-PAGE analysis) were combined and transferred to a 10,000 molecular weight cutoff (MWCO) Snakeskin dialysis tubing (Thermo Scientific) and equilibrated in dialysis buffer (50 mM K₂HPO₄ pH 8.0, 150 mM NaCl, 1 mM DTT) overnight. Dialyzed protein solutions were concentrated using a 30,000 MWCO Amicon centrifugal filter (Millipore-Sigma), and the concentrated protein solution was flash frozen as beads in liquid nitrogen (50 μL portions) and stored at −80 °C. The % FAD for each batch of protein varied and was determined by back calculation from a denaturated aliquot of enzyme using optical absorbance with the published extinction coefficient of 11,300 M⁻¹cm⁻¹ for FAD. On average, batches of Tet50, TetX7 and TetX6 ranged from 20 to 60% FAD with an average of 30% FAD for batches of Tet50, 50% FAD for batches of TetX7 and 50% FAD for batches of TetX6.

### Example 3

### Synthesis and Characterization of Probe Molecules

N-(1-Methyl)cyclopropylbenzylamine (101). To a clean, dry 100 mL round-bottom flask, equipped with a magnetic stir bar, was added 1-methylcyclopropylamine hydrochloride (108 mg, 1 mmol), K₂CO₃ (166 mg, 1.2 mmol) and 10 mL of acetone. Benzyl bromide (143 μL, 1.2 mmol) was added to the reaction mixture dropwise with stirring. The reaction mixture was allowed to stir at room temperature overnight. When the reaction was complete as determined by LC-MS analysis, the reaction mixture was filtered through a 0.45 μm PTFE syringe filter. The filtrate was concentrated under reduced pressure to provide the crude product as an oil. Final purification by RP-C18 prep-HPLC provided 101 as light-yellow oil (46 mg, 0.29 mmol, 29% yield). ¹H NMR (500 MHz, Chloroform-d): δ=7.56-7.41 (m, 2H), 7.33 (d, J=4.9 Hz, 3H), 4.03 (s, 2H), 1.45 (s, 3H), 1.04 (s, 2H), 0.48 (s, 2H). ¹³C NMR (125 MHz, Chloroform-d): δ=132.1, 130.2, 128.9, 128.7, 48.8, 35.9, 19.5, 11.6. Prep-HPLC t_{R}=6 min. MS (ESI+): [M+H]⁺; expected 162.2, found 161.9.

N-Methyl-N-benzyl-propargylamine (102). To a clean, dry 100 mL round-bottom flask, equipped with a magnetic stir bar, was added N-methylbenzylamine (130 μL, 1 mmol), K₂CO₃ (166 mg, 1.2 mmol) and 10 mL of acetone. Propargyl chloride (87 μL, 1.2 mmol) was added to the reaction mixture dropwise with stirring. The reaction was allowed to stir at room temperature overnight. When the reaction was complete as determined by LC-MS analysis, the reaction mixture was filtered through a 0.45 μm PTFE syringe filter. The filtrate was concentrated under reduced pressure to provide the crude product as an oil. Final purification by RP-C18 prep-HPLC provided 102 as colorless oil (35 mg, 0.22 mmol, 22% yield). ¹H NMR (500 MHz, Chloroform-d): δ=7.48-7.42 (m, 2H), 7.42-7.34 (m, 3H), 3.96 (s, 2H), 3.58 (d, J=2.5 Hz, 2H), 2.61 (s, 3H), 2.52 (t, J=2.5 Hz, 1H). ¹³C NMR (125 MHz, Chloroform-d): δ 134.1, 130.1, 128.8, 128.5, 76.1, 75.6, 58.6, 43.5, 40.3. Prep-HPLC t_{R}=6.0 min. MS (ESI+): [M+H]⁺; expected 160.2, found 160.2.

### Example 4

### General Procedure for the Synthesis of C9-Substituted aTC Inhibitors

To a clean, dry 25 mL round-bottom flask, equipped with a magnetic stir bar and reflux condenser, was added 1-methylcyclopropylamine hydrochloride or N-methylbenzylamine (0.3 mmol), formaldehyde (25 μL, 0.33 mmol) and 3 mL methanol. The reaction system was heated to 64 °C for 3 h. To another clean, dry 25 mL round-bottom flask, equipped with a magnetic stir bar, was added aTC (46.3 mg, 0.1 mmol) and 2 mL methanol under an argon atmosphere. The reaction system in the first flask was added to second flask dropwise using a syringe while the reaction was heated to 40 °C~0.2 mL aqueous 1 M HCl was added to dissolve all starting materials. After 16 h, the reaction progress was checked by LC-MS analysis. When the reaction was complete, the reaction mixture was concentrated under reduced pressure to yield a brown residue. The residue was dissolved in MeOH, filtered through a 0.45 μm PTFE syringe filter, and purified by RP-C18 prep-HPLC to provide the desired product as the corresponding formic acid salts. The compound was then dissolved in a minimal amount of MeOH with ~0.1 mL 1 M HCl, diluted with 20 mL of Et₂O, and cooled to 4 °C to induce precipitation of the product. After ~3 h, the solvent was removed via pipet, and the yellow solid was triturated with fresh Et₂O. The solid was dried under a stream of N2 gas as the corresponding HCL salt.

### (4S,4aS,12aS)-4-(Dimethylamino)-3,10,11,12a-tetrahy-droxy-6-methyl-9-(((1-methylcyclopropyl)amino)methyl)-1,12-dioxo-1,4,4a,5,12,12a-hexahydrotetracene-2-carboxamide Hydrochloride (103)

103 was prepared according to the general procedure and was obtained in 69% yield as a yellow solid. ¹H NMR (600 MHz, DMSO-d6) δ 7.88-7.94 (d, 1H)^{a}, 7.51-7.56 (d, 1H)^{a}, 4.92-4.94 (d, 1H), 4.28 (s, 2H), 3.54-3.59 (m, 1H), 3.51-3.54 (m, 1H), 2.95-3.01 (m, 11H), 2.90 (s, 6H), 2.37-2.44 (s, 3H)^{b}, 1.51 (s, 3H), 1.12-1.15 (t, 2H), 0.65-0.68 (t, 2H). ¹³C NMR (150 MHz, DMSO-d6): δ 188.0, 162.9, 156.2, 139.1, 135.3, 131.8, 122.2, 115.1, 113.8, 111.6, 109.0, 76.3, 67.0, 66.0, 42.3, 38.6, 36.5, 26.3, 18.3, 14.0, 10.8. Prep-HPLC t_{R}=11.0 min. MS (ESI+): [M+H]⁺; expected 510.2, found 510.2. High-resolution mass spectrometry (HRMS) (time-of-flight (TOF) MS ESI+): calcd for C₂₇H₃₁N₃O₇ [M+H]⁺, 510.2240; found, 510.2227. ^{a}Appeared as two doublets, indicating the formation of the epimer during the synthesis. ^{b}Appeared as two singlets representing the methyl group from each epimer respectively.

### (4S,4aS,12aS)-4-(Dimethylamino)-3,10,11,12a-tetrahydroxy-6-methyl-1,12-dioxo-9-((prop-2-yn-1-ylamino)-methyl)-1,4,4a,5,12,12a-hexahydrotetracene-2-carboxamide Hydrochloride (104)

Compound 104 was prepared according to general procedure and was obtained in 30% yield as a yellow solid. ¹H NMR (600 MHz, DMSO-d6) δ 7.81-7.86 (d, 1H)^{a}, 7.51-7.56 (d, 1H)^{a}, 4.92-4.94 (d, 1H), 4.30 (s, 2H), 3.90-3.92 (d, 2H), 3.54-3.59 (m, 1H), 3.51-3.54 (m, 1H), 2.95-3.01 (m, 1H), 2.90 (s, 6H), 2.37-2.44 (s, 3H)^{b}. ¹³C NMR (150 MHz, DMSO-d6): δ 189.9, 188.0, 156.2, 139.1, 134.7, 122.2, 115.2, 113.2, 111.5, 79.6, 77.8, 77.0, 75.0, 66.0, 64.9, 43.3, 38.6, 35.5, 26.4, 14.0. Prep-HPLC t_{R}=10.2 min. MS (ESI+): [M+H]⁺; expected 494.2, found 494.2. High-resolution mass spectrometry (HRMS) (time-of-flight (TOF) MS ESI+): calcd for C₂₆H₂₇N₃O₇ [M+H]⁺, 494.1927; found, 494.1909. ^{a}Appeared as two doublets, indicating the formation of the epimer during the synthesis. ^{b}Appeared as two singlets representing the methyl group from each epimer respectively.

### 4S,4aS,12aS)-4-(Dimethylamino)-3,10,11,12a-tetrahy-droxy-6-methyl-9-((methyl(prop-2-yn-1-yl)amino)methyl)-1,12-dioxo-1,4,4a,5,12,12a-hexahydrotetracene-2-carboxamide (105)

Compound 105 was prepared according to general procedure and was obtained in 29% yield as a yellow solid. ¹H NMR (300 MHz, Methanol-d4) δ 7.73 (d, J=8.8 Hz, 1H), 7.61 (d, J=8.6 Hz, 1H), 4.65 (dd, J=12.9, 3.8 Hz, 1H), 4.50 (dd, J=12.9, 4.0 Hz, 1H), 4.28 (d, J=3.1 Hz, 1H), 4.16 (d, J=6.7 Hz, 2H), 3.68 (dd, J=17.7, 4.8 Hz, 1H), 3.52-3.45 (m, 1H), 3.44 (s, 1H) 3.13-3.09 (d, 1H), 3.08 (s, 6H), 2.96 (s, 3H), 2.48 (s, 3H). ¹³C NMR (150 MHz, Methanol-d4): δ 160.0, 137.3, 134.8, 127.7, 125.6, 118.5, 113.4, 111.8, 82.9, 80.5, 79.2, 74.3, 71.0, 56.3, 47.4, 45.5, 44.3, 41.8, 38.5, 31.7, 15.7. Prep-HPLC t_{R}=10.7 min. MS (ESI+): [M+H]⁺; expected 508.5, found 508.7.

### Example 5

### Optical Absorbance Spectroscopy and LC-MS Detection of Covalent FAD Adducts

A solution of 20 μM Tet50 (final protein concentration based on A₂₈₀; working concentration of active enzyme was predicted to be 4-12 μM based on FAD content) in the presence of 6 mM probe, either 101 or 102, in 100 mM TAPS buffer (pH=8.5) in polystyrene cuvettes was analyzed by scanning optical absorbance (280-550 nm) using the Agilent Cary 50 UV-visible spectrophotometer. The unexposed ‘dark’ samples were protected from external light by keeping the cuvettes inside the spectrophotometer throughout the entire data collection period. Absorbance spectra (280-550 nm) were acquired at 1 min interval for a total of 10 min. For the ‘exposed’ samples, the cuvette was removed from the spectrophotometer between spectral acquisitions (1 min) and illuminated by a 700 Im blue LED bulb from a distance of 20 cm for 45 s (cumulative light exposure time of 7.5 min). All data generated from the UV-vis spectrophotometer were replotted with GraphPad Prism 10 software. Consecutive acquisitions were stacked in one graph with gradient blue lines (from 0 to 10 min, darkest to lightest).

The samples after spectral acquisition under blue light exposure were then quenched with enough methanol to denature the enzymes. The solid precipitates were removed by centrifugation at 13,000 rpm for 5 min. The supernatant was removed, filtered through a 0.2 μm syringe filter, and analyzed by LC-MS and HRMS to detect the covalent FAD adducts.

### Example 6

### Time Dependence of TDase Inhibition

TDase activity was determined from the apparent velocities of TC degradation as judged by a decrease in A₄₀₀ over time with variable enzyme-inhibitor preincubation times and in the presence of varying concentrations of inhibitors at room temperature. PHBH activity was determined similarly using para-hydroxybenzoic acids as substrate and monitoring for NADPH consumption via A₃₄₀. Reaction samples were prepared in 100 mM TAPS buffer (pH 8.5) with 500 μM NADPH, 5 mM MgCl₂, 100 μM TC, varying concentrations of inhibitor, and 1 μM TDase (final protein concentration judged by A₂₈₀; working concentration of active enzyme was predicted to be 0.2-0.6 μM based on FAD content). Tet50 (1 μM final concentration) was incubated with inhibitors at final working concentrations of 0, 4, 8, 16, 31, 63, 125, 250, and 500 μM. TetX7 (1 μM final concentration) was incubated with inhibitors at final working concentrations of 0, 0.5, 1, 2, 4, 8, 16, 32, and 64 μM. Reaction samples were prepared in a Costar 96-well flat bottom clear, polystyrene plate and incubated under dark or a 700 lm blue LED bulb held at a distance of 20 cm for various durations. Incubation times were 0, 30, 60, 120, and 240 min under dark or 0, 1, 2, 4, and 8 min under blue light. Following incubation, the enzyme-inhibitor samples were diluted 2-fold with substrate solution containing TC and NADPH, and the initial rate was measured by monitoring optical absorbance spectroscopy at 400 nm for 3 min. Initial enzyme velocities were determined by linear regression over the linear range of the reaction (typically between 0 and 1 min). The rates were expressed as percent activity remaining by normalizing to the rate of TDases that had not been treated with inhibitors. The velocities were plotted against the logarithm of inhibitor concentration, and apparent IC₅₀ plots were analyzed using GraphPad prism 10 software. The apparent IC₅₀ values from 0 min incubation were summarized in Table 1 (see below) as an indicator of the inhibitors' reversible binding affinity compared with substrates. All apparent IC₅₀ values were plotted against the duration of incubation time and fitted to an exponential decay function. All data points were shown as means and error bars represented standard deviation at least two independent experiments.

### Example 7

### Testing the Reversibility of TDase Inhibition by Exhaustive Dialysis

The reversibility of TDase inhibition was directly assessed by exhaustive dialysis of TDase-inhibitor complexes followed by analysis in the in vitro activity assay. TDases (40 mL of 110 mM protein stock concentration as judged by A₂₈₀; working concentration of active enzyme was predicted to be 22-66 μM based on FAD content) were mixed with 760 mL of inhibitor stock solution, 1.64 mM stock for Tet50 or 0.49 mM stock for TetX7, targeting a final concentration of inhibitor near the predetermined IC₅₀ values of 284 μM for Tet50 or 45 μM for TetX7 (see Table 1 below).

TABLE 1
Apparent IC₅₀ Values of Compounds 101 to 106
Measured through In Vitro Inhibition of the Oxidation
of Tet by Tet50, TetX7, and TetX6 under Dark Conditions

| | Apparent IC₅₀ values (μM) | | |
|---|---|---|---|
| Inhibitor | Tet50 | TetX7 | TetX6 |
| 101 | None detected^{a} | None detected^{a} | None detected^{a} |
| 102 | None detected^{a} | None detected^{a} | None detected^{a} |
| 103 | 430 ± 40 | 11 ± 5 | 50 ± 4 |
| 104 | 600 ± 40^{b} | 74 ± 8 | 69 ± 7 |
| 105 | 470 ± 60^{b} | 70 ± 10 | Not tested |
| 106 | 410 ± 50 | 40 ± 6 | 33 ± 3 |

^{a}Inbition was not detected for concentrations of compounds 101 and 102 up to 2.5 mM.
^{b}30 min preincubation of inhibitor with enzyme prior to initiating assay

The mixtures were incubated in the dark for 60 min or under blue light for 15 min to induce covalent inhibition at room temperature. The samples were dialyzed in a 50 mL Thermo Scientific Slide-A-Lyzer MINI Dialysis Devices, 10K MWCO overnight (~18 h) at 4 °C against 100 mM TAPS buffer (pH 8.5). TDase activity was determined before and after dialysis by addition of 200 mL enzyme-inhibitor preincubation mixture to 900 mL reaction mixture prepared in 100 mM TAPS buffer (pH 8.5) with 500 μM NADPH, 5 mM MgCl₂, and 100 μM TC (all final concentration) in a polystyrene cuvette (1 mL working volume). Reaction progress was monitored at A₄₀₀ (decrease in TC absorbance band) for 3 min using the Agilent Cary 50 UV-visible spectrophotometer. Initial enzyme velocities were determined by linear regression using Agilent Cary WinUV Software over the linear range of the reaction (typically between 0 and 1 min). The remaining enzymatic activity was normalized as a percent of the noninhibited controls. All data points were shown as means and error bars represent standard deviations from three independent experiments.

### Example 8

### Minimum Inhibitory Concentration (MIC)

MIC panels were prepared in 96-well flat-bottom microplates (Corning) by 2-fold serial dilution of inhibitors in cation-adjusted MH-II broth (BD) supplemented with 50 μg/mL kanamycin (KAN50), 1 mM IPTG, and with or without 16 μg/mL of TC. The panels were stored at −80 °C before use. Single colonies of E. coli DH5αZ1 containing TetX7, Tet50, or empty vector in pZE24 expression system were grown in MH-II broth with KAN50 and growth for overnight at 37 °C. On the day of the experiment, MIC panels were thawed at room temperature. Overnight cultures were subcultured in fresh MH-II broth with KAN50 and 1 mM IPTG, grown to exponential phase (OD600 of 0.3-0.8), then diluted in MH-II broth with KAN50 and 1 mM IPTG. The diluted cells were inoculated into the MIC panel at a 1:1 ratio, resulting in a final concentration of ~5×10⁵ CFU/mL cells in each well. The panels were incubated at 37 °C for 20 h, then scored by visual inspection. Potential synergistic effects between inhibitors and TC were evaluated by comparing the MIC of inhibitors alone to the MIC of inhibitors in combination with TC. Each test was performed in triplicate, with no-antibiotic and no-cell control wells.

### Example 9

### Comparison of the In Vitro Photostability of FMOs

For tetracycline destructases (TDases), reactions (in triplicate) were prepared in 100 mM TAPS buffer (pH 8.5) with 500 mM NADPH, 5 mM MgCl₂, 100 mM tetracycline, and 1 mM enzyme (final protein concentration; the working concentration of the active enzyme was predicted to be 0.2-0.6 μM based on FAD content). The reaction samples containing only the enzyme were incubated under blue light for 0, 1, 2, 4, or 8 min. Following incubation, the substrates were added to the reaction samples to initiate the reaction. The reaction was monitored by UV-visible spectroscopy at 400 nM for 3 min. The initial enzyme velocities were determined via linear regression over the linear range of the reaction (typically between 0 and 1 min). The rates were expressed as the present activity remaining by normalizing to the rate of TDases that had 0 min of light incubation. The remaining enzyme activity values were plotted against the duration of light incubation time. Means and standard errors from at least two independent experiments were calculated.

For para-hydroxybenzoate hydroxylase (PHBH), the same procedure was conducted without monitoring the reactions at 340 nm and changing the substrates and concentrations to 200 mM NADPH, 2 mM MgCl₂, 125 mM para-hydroxybenzoate acid, and 0.5 mM enzyme.

### Example 10

### Spectroscopic Characterization of the Light-Dependent Inactivation of PHBH by Compounds 101 and 102

The spectra of 20 μM PHBH in the presence of 6 mM 101 or 102 were measured in polystyrene cuvettes using an Agilent Cary 50 UV-visible spectrophotometer. To obtain spectra of dark incubation control, the sample was protected from external light by keeping the cuvette inside the spectrophotometer throughout 12 h of spectra acquisition at 30 min intervals. To obtain the spectra of the covalently inactivated enzyme, the cuvette was removed from the spectrophotometer between spectral acquisitions (1 min) and exposed to a 700-lumen blue LED bulb from a distance of 20 cm for 45 s (cumulative light exposure time of 7.5 min).

### Example 11

### Cyclopropylamine and Propargylamine Warheads Covalently Tag FAD in TDases

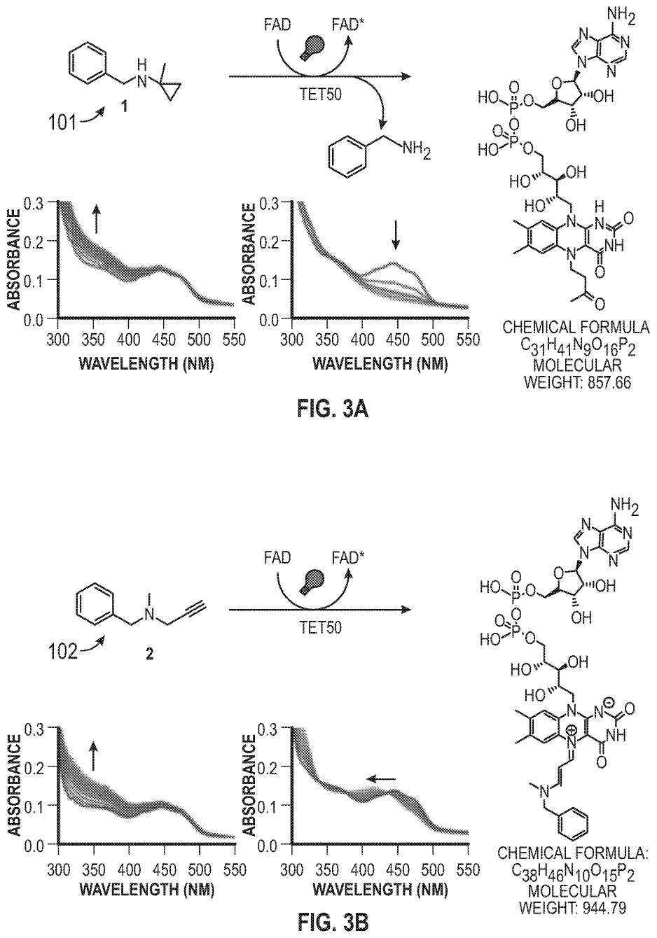

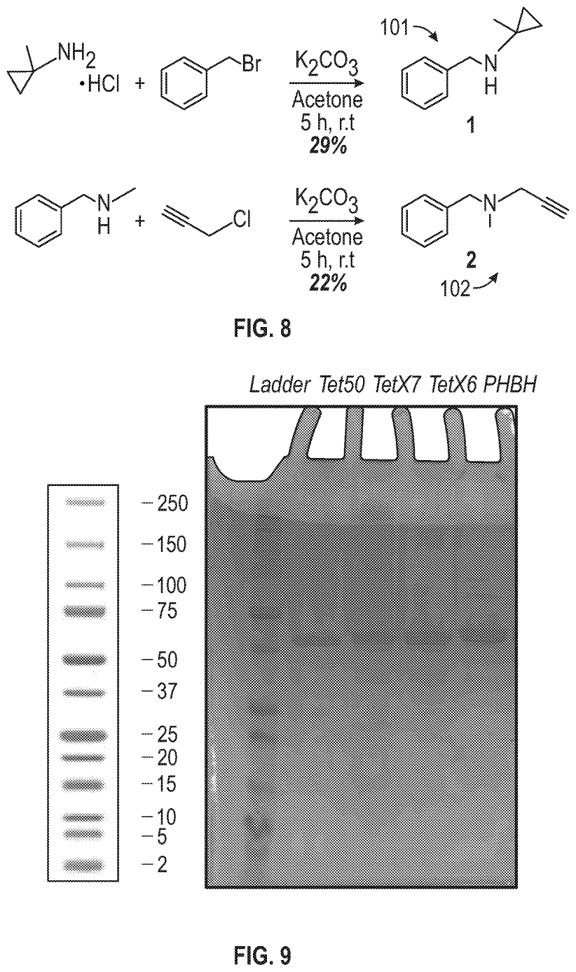

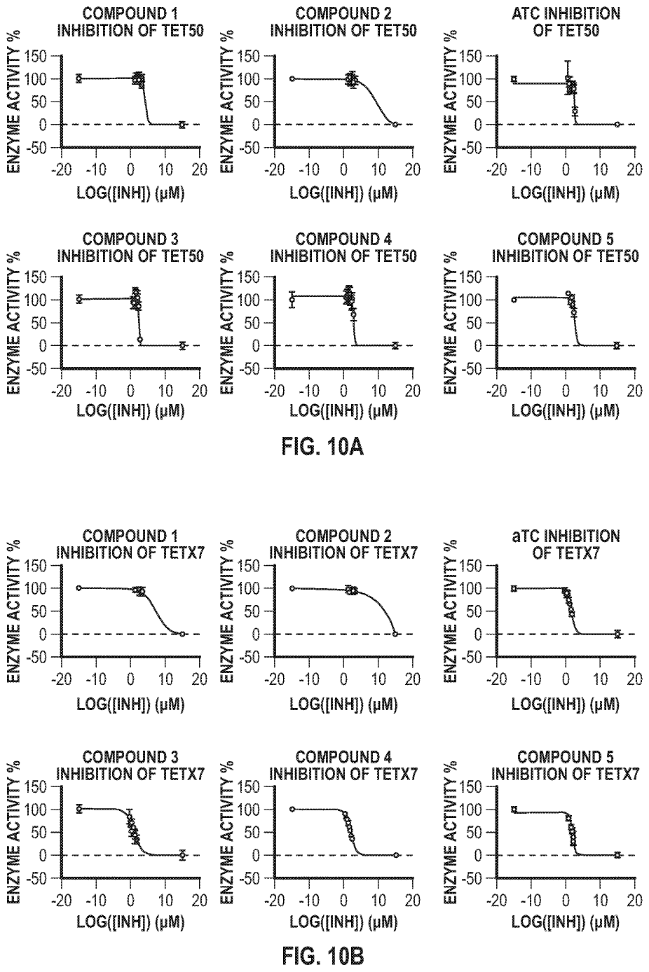

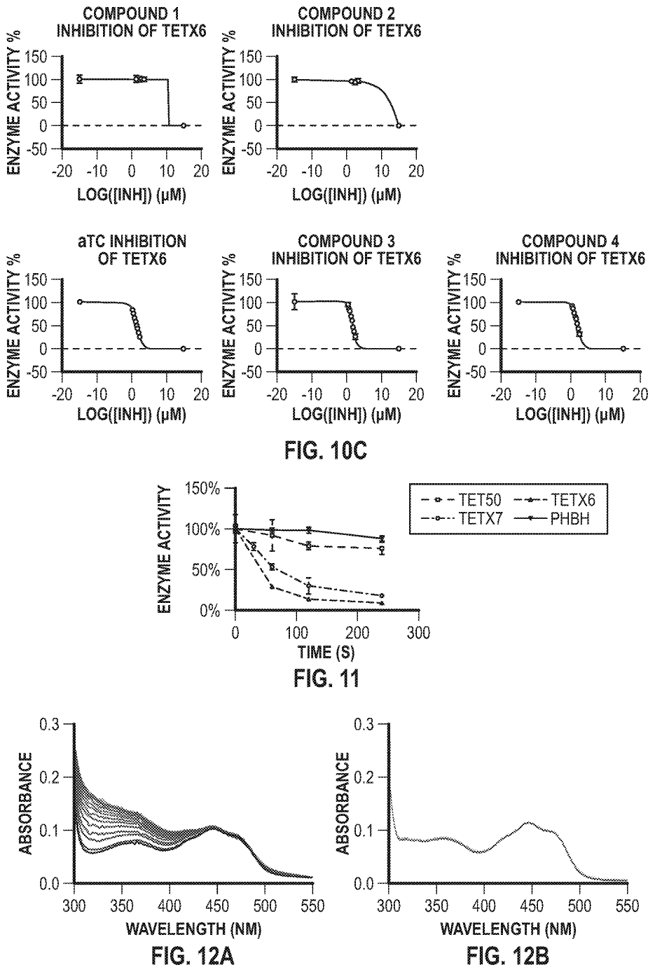

Two mechanistic probe molecules, N-(1-methyl)cyclopropylbenzylamine 101 and N-methyl-N-benzyl-propargylamine 102 (FIG. 3), were used to test whether cyclopropylamine and propargylamine warheads were capable of reacting with the FAD cofactors of TDases. 101 was synthesized by S_{N}2 alkylation of 1-methylcyclopropylamine with BnBr under basic conditions (FIG. 8). Probe 102 was synthesized by S_{N}2 alkylation of N-Me-benzylamine with propargyl chloride under basic conditions. Heterologous expression of TetX6 (Type 1 TDase), TetX7 (Type 1 TDase), Tet50 (Type 2 TDase), and 4-hydroxybenzoate-3-monooxygenase (PHBH; a control class A FMO) provided the purified enzymes that were used for in vitro enzyme assays (FIG. 9). A TC consumption assay was used to measure TDase activity by optical absorbance at 400 nm (λₘₐₓ TC). No inhibition of TDase activity was detected at concentrations of the probe molecules 101 and 102 up to 2.5 mM (Table 1 and FIG. 10). Blue light exposure was used to enhance the reactivity of the FAD cofactor toward the probe molecules. The Type 1 TDases TetX6 and TetX7 lost ~91% and 82% of their apparent activity, respectively, after 4 min of exposure to blue light. However, the Type 2 TDase Tet50 and control FMO PHBH maintained ~76%±3% and 88%±3% apparent activity, respectively, under the same conditions (FIG. 11). Hence, Tet50 and PHBH were chosen for further study with molecules 101 and 102 under blue light irradiation.

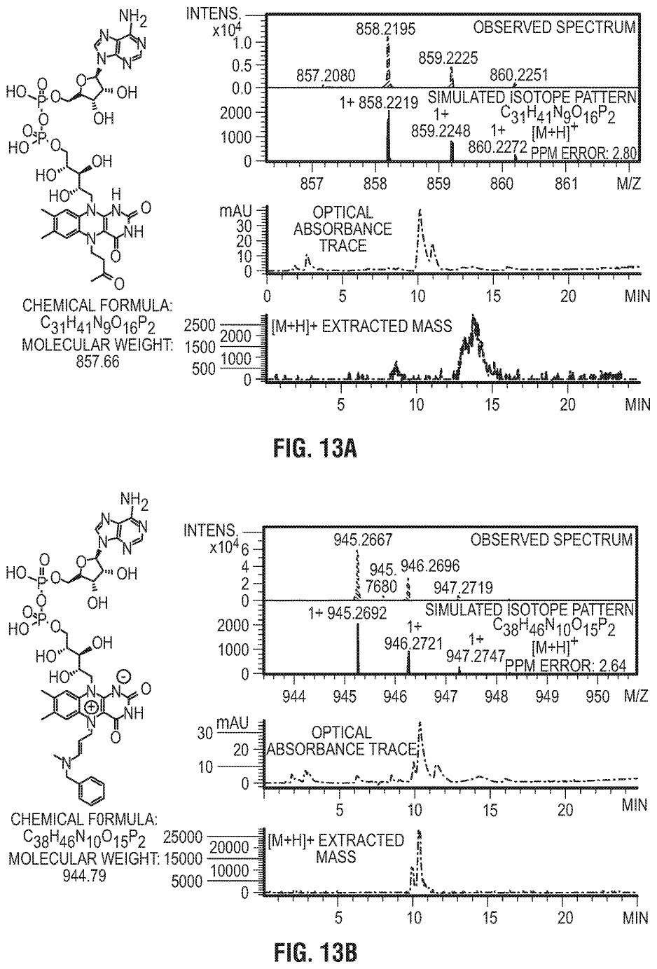

Using a higher concentration of Tet50 allowed for the clear visualization of two major absorbance bands centered at ~365 nm and ~450 nm which were characteristic of an oxidized flavoenzyme spectrum (FIG. 3). Under dark incubation using Tet50 in the presence of 101 and 102, there was a significant absorbance increase at 365 nm, which also appears in the absorbance spectra of the enzyme alone control (FIG. 3 and FIG. 12). However, treatment of Tet50 with cyclopropylamine (101) under blue light irradiation resulted in a continuous decrease in the absorbance of the bands at 450 nm (FIG. 3). Visual inspection of the cuvette revealed near complete quenching of the canonical yellow color of the flavoenzyme solution. These spectral changes were indicative of the reduction of FAD with alkylation at the N5 position of the flavin isoalloxazine ring. The resulting absorbance spectrum was remarkably similar to the reported absorbance spectra of MAOs after treatment with the cyclopropylamine inhibitor tranylcypromine. To confirm the identity of the FAD adduct, high-resolution mass spectrometry (HRMS) was performed on the treated and control samples with an unbiased comparison of the molecular ion profiles. A denatured sample of Tet50 treated with cyclopropylamine (101) under blue light irradiation produced a unique molecular ion with an observed m/z value of 858.2195 correlating within 2.80 ppm error to the expected m/z value of 858.2219 calculated for the [M+H]⁺ ion of the 4-(flavin-N5-yl)-butanone adduct shown in FIG. 3 (see also FIG. 13).

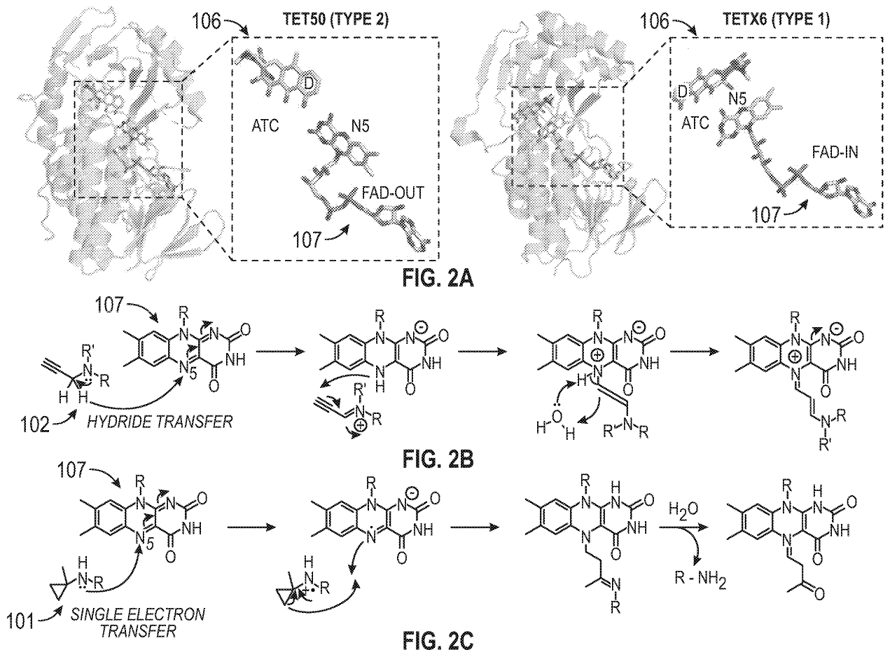

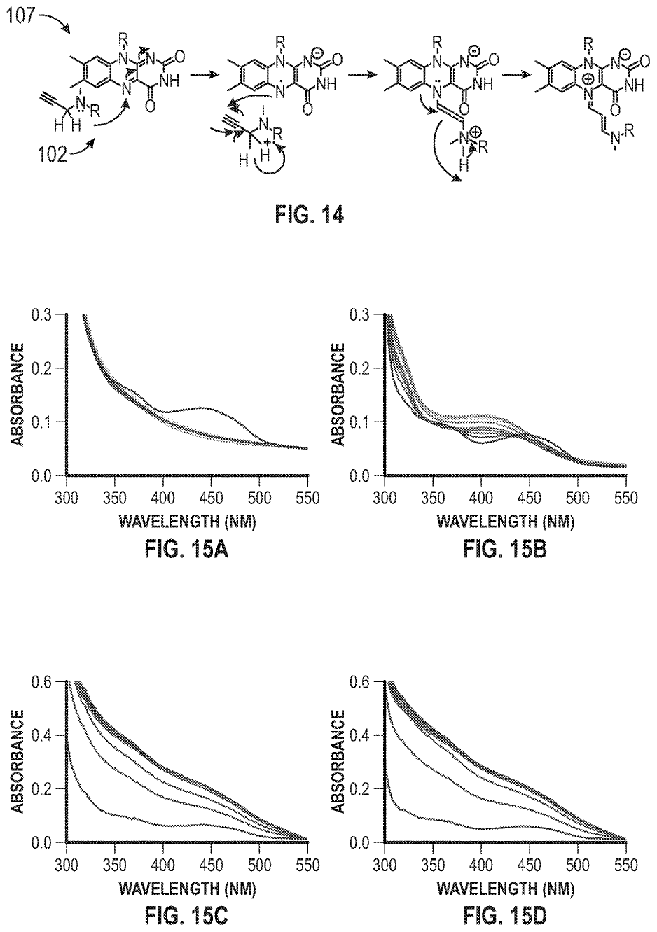

The incubation of Tet50 with propargylamine (102) under blue light irradiation resulted in an increase in absorbance at 320 nm, a decrease in absorbance peak at 365 nm, and a blueshift in the second absorbance peak from ~450 nm to ~414 nm indicating the formation of a new conjugated FAD species (FIG. 3). Visual inspection of the reaction cuvette revealed that the characteristic yellow color of a flavoenzyme was maintained in this sample. These spectral changes were indicative of the reduction of FAD with iminium ion formation at the N5 position of the flavin isoalloxazine ring with strong similarity to the absorbance spectra of MAOs after treatment with the propargylamine inhibitor propargyline. A denatured sample of Tet50 treated with propargylamine under blue light irradiation produced a unique molecular ion in the HRMS spectra with an observed m/z value of 945.2667 correlating within 2.64 ppm error to the expected m/z value of 945.2692 calculated for the [M+H]⁺ ion for the (E)-4-(3-(benzyl-(methyl)amino)allylidene)-N5-flavin-4-ium-4-ide adduct shown in FIG. 3 (see also FIG. 13). While propargylamine inhibitors of flavoenzymes were proposed to form this adduct via a two-electron mechanism (FIG. 2), under blue light irradiation it was also possible to propose a one-electron mechanism leading to this same FAD adduct (FIG. 14). The same pattern of reactivity was observed against PHBH (FIG. 15).

### Example 12

### Structure-Based Design of Covalent TDase Inhibitors

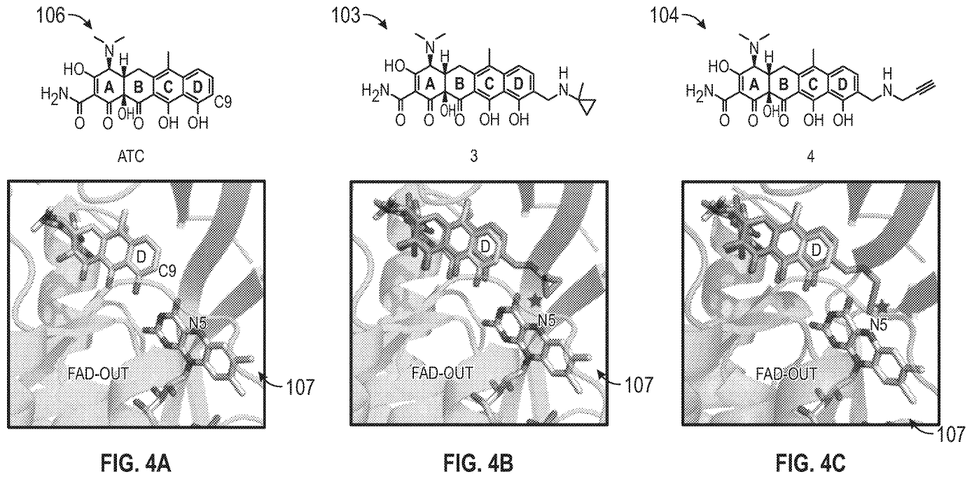

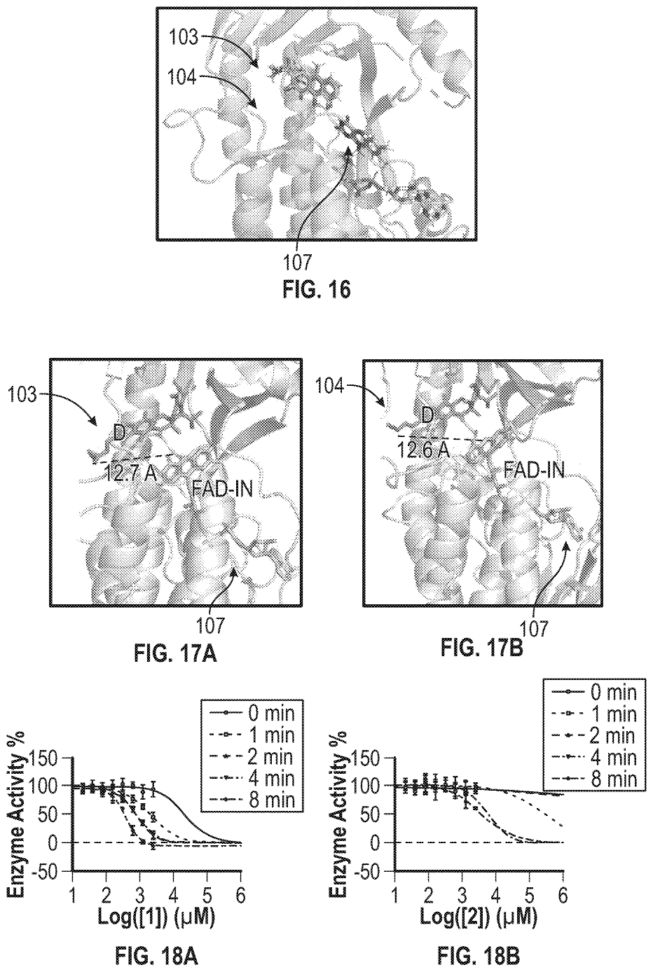

Structure-based molecular modeling was used to design more specific covalent TDase inhibitors bearing cyclopropylamine and propargylamine warheads. Known TDase inhibitor aTC was selected as the template scaffold for the covalent TDase inhibitors. aTC was a reversible inhibitor of both Type 1 and Type 2 TDases; however, the inhibitor binding modes observed in X-ray cocrystal structures with Type 1 and Type 2 TDases were distinct. For Type 1 TDases, the aTC:TetX6 structure (PDB: 8ER0) showed that aTC adopts a substrate-like binding mode with the FAD-IN conformation (FIG. 2). For Type 2 TDases, the aTC:Tet50 structure (PDB: 5TUF) showed a novel binding mode with the FAD-OUT conformation (FIG. 4). A template model based on this published crystal structure recapitulated the crystallographically observed aTC binding mode in Tet50 using molecular docking in AutoDock Vina (FIG. 16). Molecular modeling suggested that the attachment of a cyclopropylamine (compound 103, FIG. 4) or propargylamine (compound 104, FIG. 4) warhead to the C9 position of the aTC D-ring via a methylene linker was able to orient the warhead favorably for reaction with the FAD cofactor. Based on the proposed mechanisms shown in FIG. 2, the reactive atoms in the cyclopropylamine and propargylamine side chains of compounds 103 and 104 were within 4.7 Å and 4.3 Å of the N5 atom of the FAD isoalloxazine ring system, respectively. Attempts to dock a N-methylpropargylamine derivative (compound 105) failed to recapitulate the aTC binding mode in Tet50 because of a steric clash within the residues that line the solvent exposed channel accessed by the C9-substituent. It was unknown whether aTC-based inhibitors can access this ligand binding mode in Type 1 TDases. However, it was clear that there was ample space for accommodation of the C9-side chains in compounds 103 and 104 should the substrate binding mode observed in the aTC:TetX6 structure (PDB: 8ER0) be adopted. This binding mode was predicted to position the reactive warheads far away from the FAD cofactor (>12 Å; FIG. 17).

### Example 13

### Synthesis of Covalent TDase Inhibitors

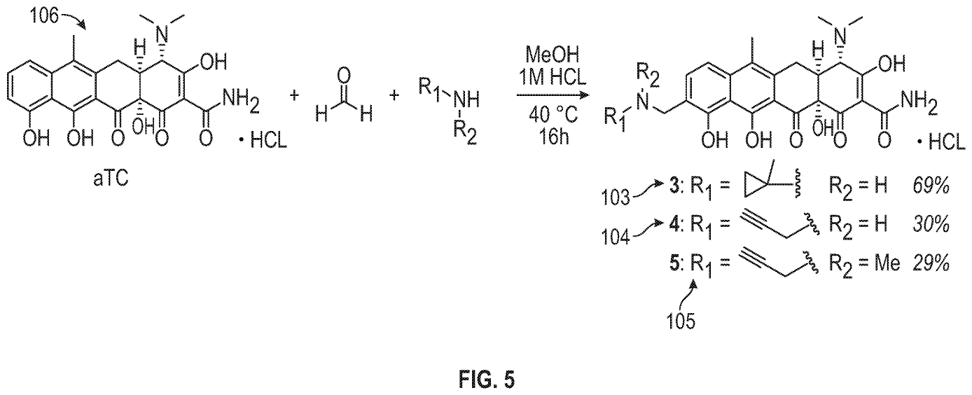

Prior semi-syntheses of aTC derivatives demonstrated that selectivity can be achieved through aromatic electrophilic substitution at the C9 position of the electron-rich D-ring. Notably, both reactive warheads earmarked for installation were derived from primary amine precursors. The Mannich reaction between alkylamines, aldehydes, and TC served as an exceptionally efficient approach for preparing C9-aminomethylcycline derivatives of TC including the FDA-approved third-generation drug omadacycline. Hence, it was determined that the Mannich reaction was able to be applied to the synthesis of C9-aminomethylcycline derivatives of aTC. Specifically, either 1-methylcyclopropylamine hydrochloride or propargylamine was treated with 1.1 equiv of formaldehyde in methanol under refluxing conditions to generate the corresponding Schiff base. This mixture was combined with 0.2 equiv of aTC in 1 M HCl_{aq} and stirred for 16 h at 40 °C to provide the corresponding Mannich products, 103, 104, and 105 (FIG. 5). The final purification of compounds 103, 104, and 105 was achieved via semipreparative RP-C18 HPLC resulting in apparent yields of 69%, 30%, and 29%, respectively. The structures were confirmed through multidimensional NMR analysis. Signals from the aTC scaffolds were consistent with previous reporting. The side chain connection on C9 was proven by HMBC correlation between C10 (~155-160 ppm) and the methylene linker.

### Example 14

### In Vitro Inhibition of TDases

The inhibitory properties of compounds 103, 104, and 105 toward the TDase-catalyzed oxidation of TC were evaluated using a steady-state kinetic assay monitored continuously by optical absorbance (A₄₀₀ tracks the disappearance of TC). This in vitro assay was used to determine the apparent IC₅₀ values for TDase inhibitors under a fixed concentration of TC, NADPH, and O₂. TetX7 and TetX6 as model Type 1 enzymes and Tet50 as a model Type 2 enzyme were used. TDase inhibitor aTC (106) was used as a positive control. It was found that 106, 103, 104, and 105 were ~10-fold more potent inhibitors of TetX7 and TetX6 than Tet50 (Table 1). This preference for the inhibition of Type 1 TDases over Type 2 TDases was consistent with prior reports of aTC analogs. The apparent IC50 values were in line with those of the parent inhibitor aTC indicating that substitution by methylene-linked cyclopropylamine or propargylamine at the C9 position of the aTC D-ring was tolerated by both Type 1 and 2 TDases.

### Example 15

### Time- and Light Dependence of TDase Inhibition

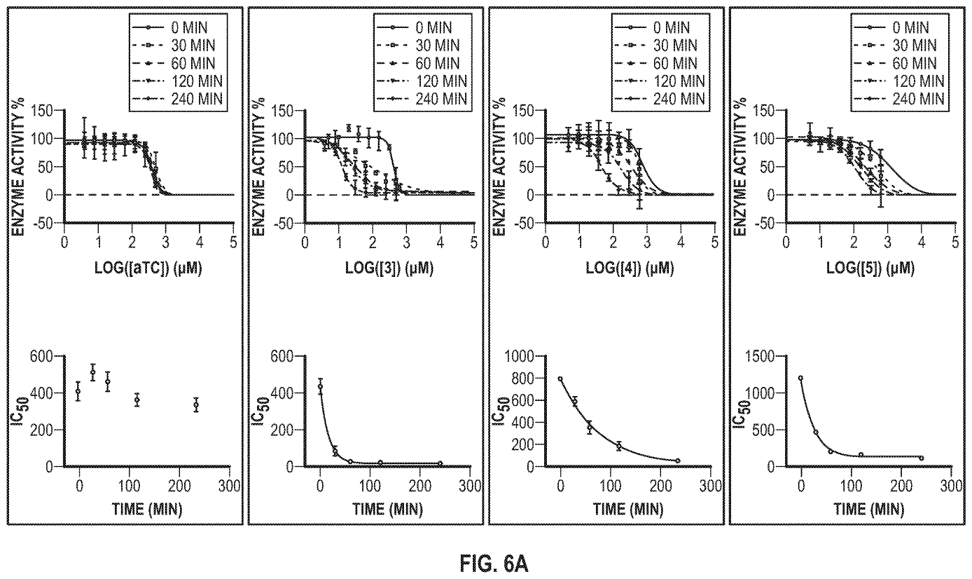

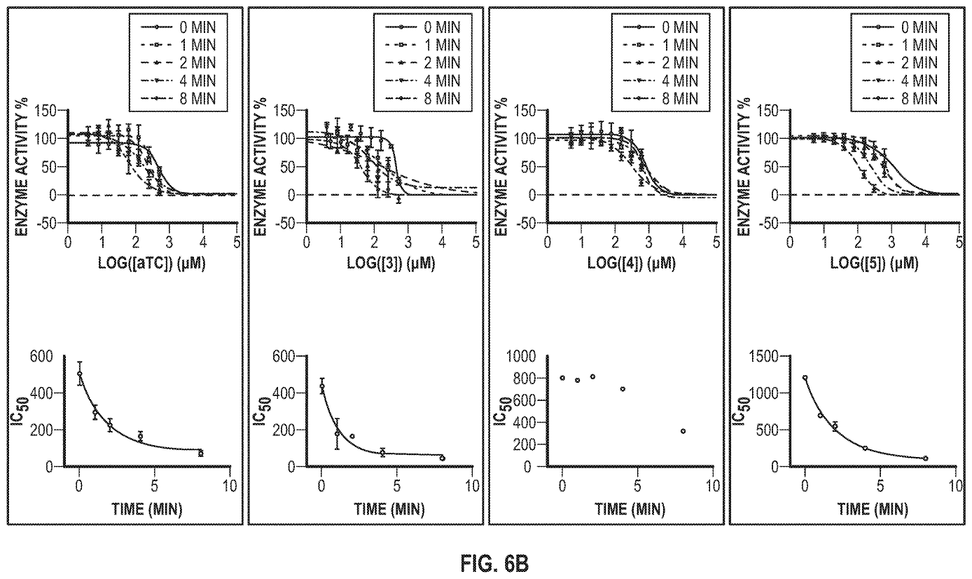

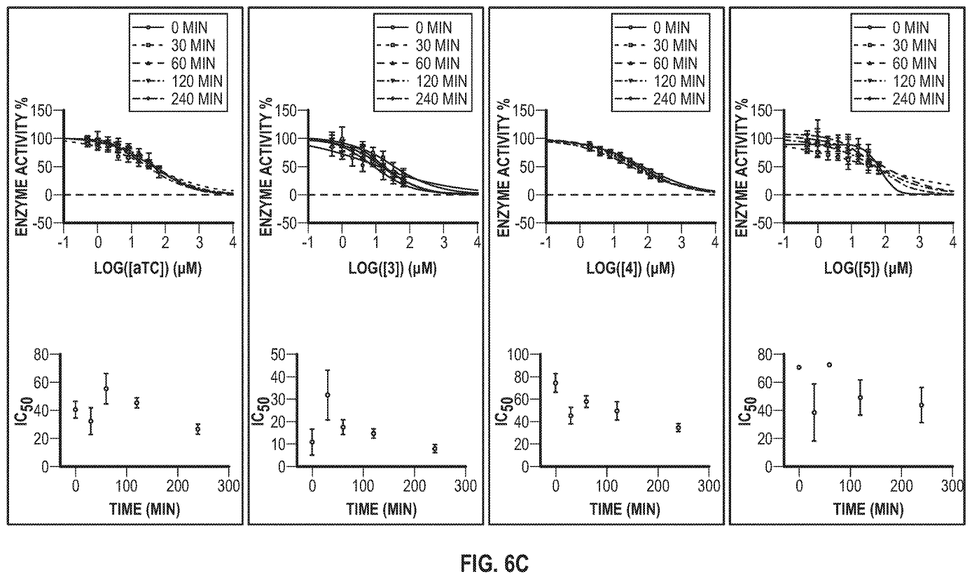

A hallmark of covalent enzyme inhibition was time dependence given that longer exposure of the enzyme to the inhibitor allowed for more inactivating turnovers. Tet50 was incubated with an inhibitor, either 106, 103, 104, or 105, for 0, 30, 60, 120, or 240 min prior to initiating the TDase reaction by the addition of TC and NADPH. The apparent IC₅₀ values were determined for each preincubation time point by monitoring the TDase velocity at A₄₀₀ under various inhibitor concentrations. The apparent IC₅₀ values were plotted against the preincubation times to determine if the TDase inhibition was time-dependent (FIG. 6). When the reactions were shielded from light, the inhibition of Tet50 by aTC (106) was not time-dependent, whereas 103, 104, and 105 all showed a clear decrease in the apparent IC₅₀ value correlating to longer enzyme-inhibitor preincubation times. The apparent IC₅₀ values for aTC remained constant between 410±50 μM and 340±40 μM over 240 min. The apparent IC₅₀ values for compounds 103, 104, and 105 decreased from 430±40 μM to 13±1 μM, 600±40 μM to 48±7 μM, and 470±60 μM to 70±20 μM respectively, over 240 min. These data were fit to a single-phase exponential decay function with apparent decay constants of 0.061±0.003 min⁻¹, 0.013±0.002 min⁻¹, and 0.040±0.014 min⁻¹ for 103, 104, 105, respectively. The apparent half-life values for the decay functions were 11.3 min (95% CI: 2.8-33.7 min), 54.5 min (95% CI: 32.3-175.9 min), and 18.9 min (85% CI: 10.1-141.3 min) for 103, 104, 105, respectively.

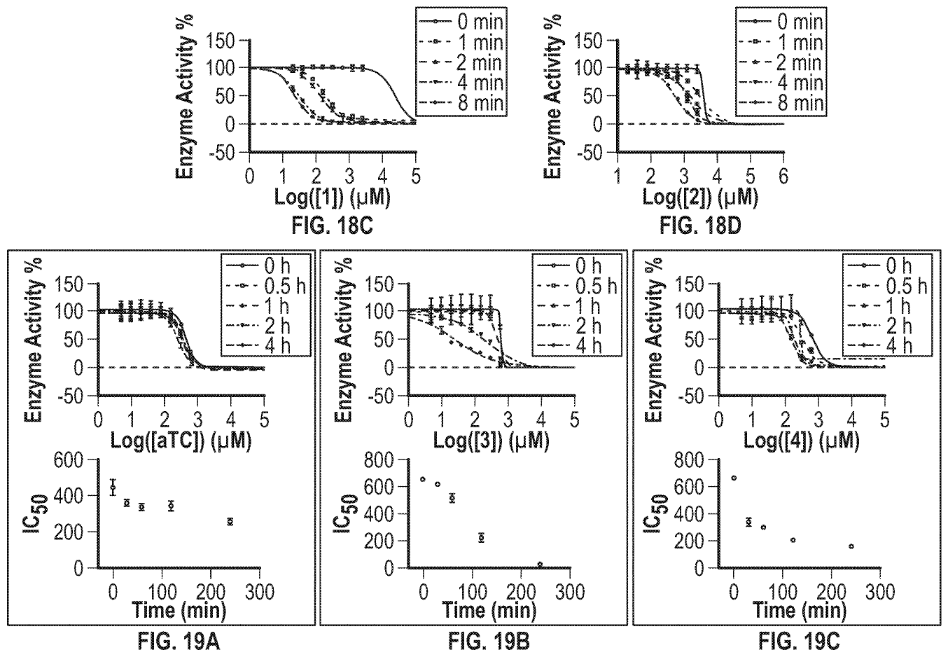

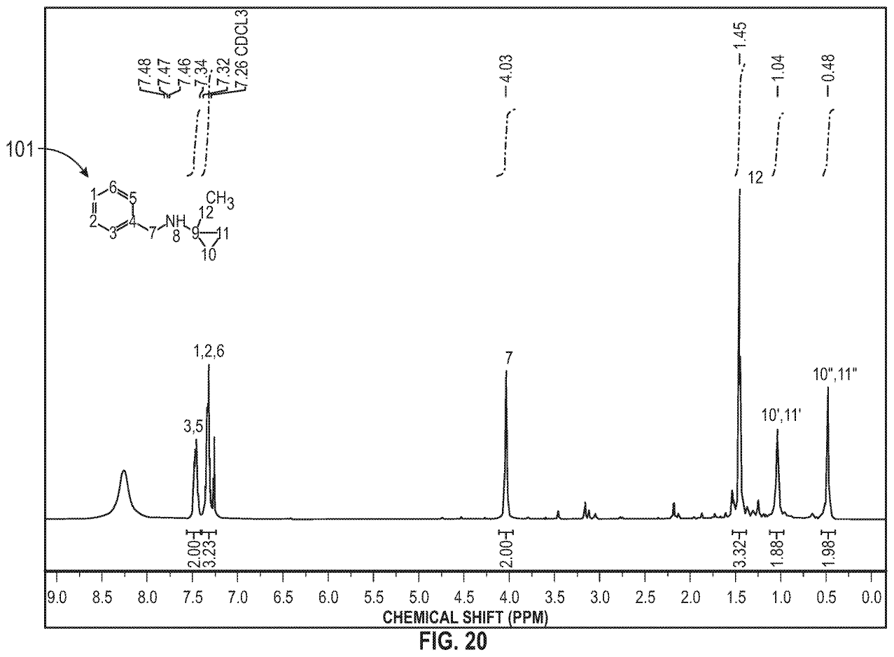

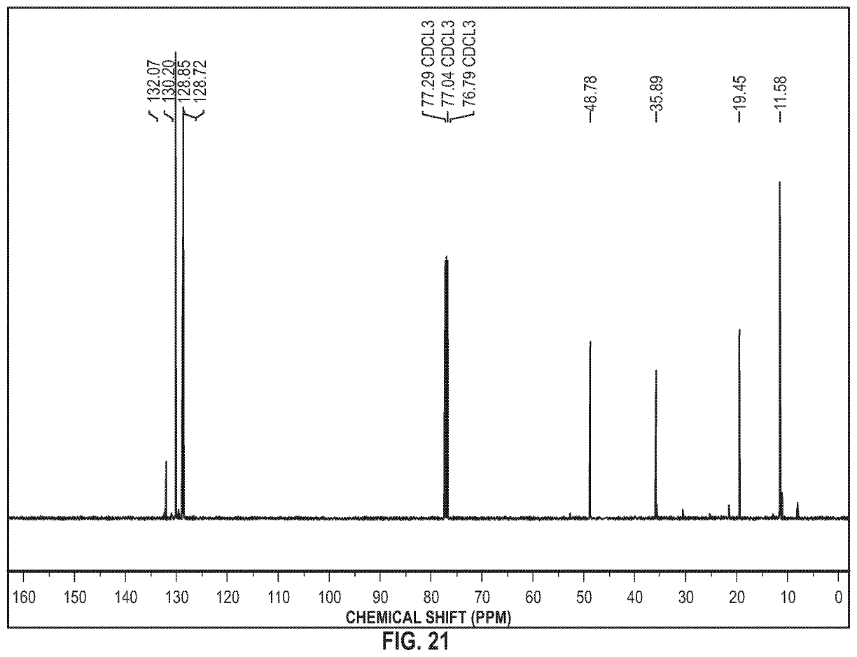

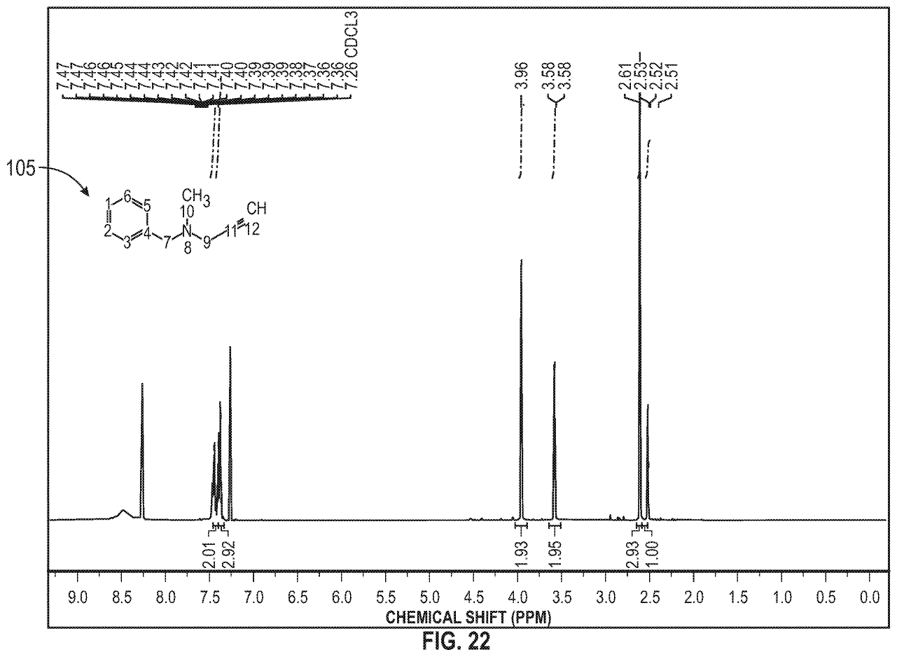

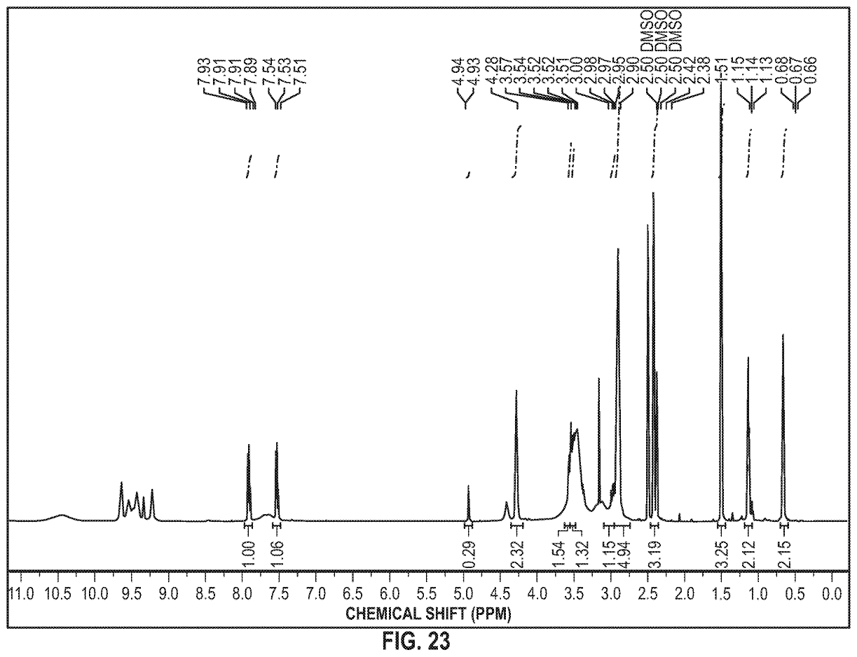

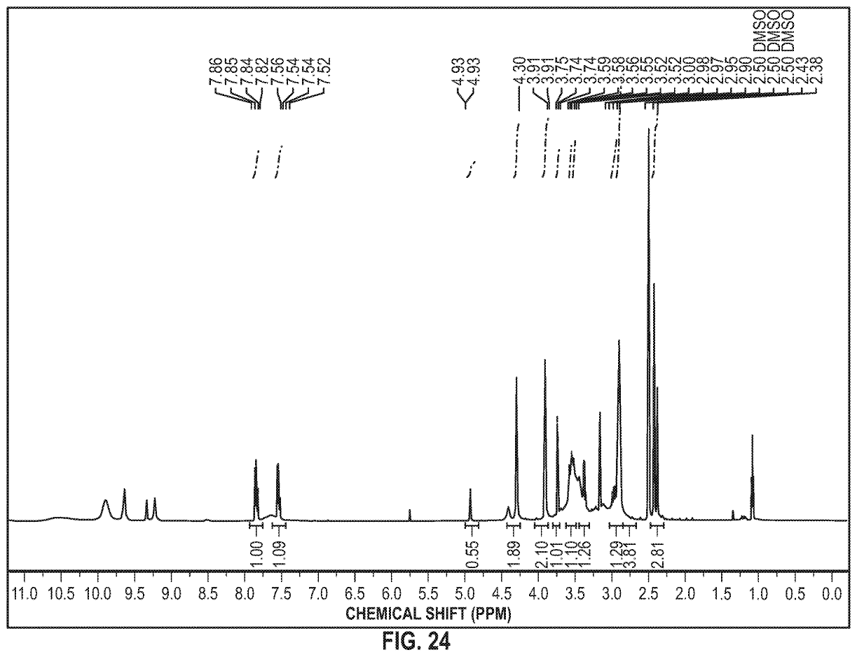

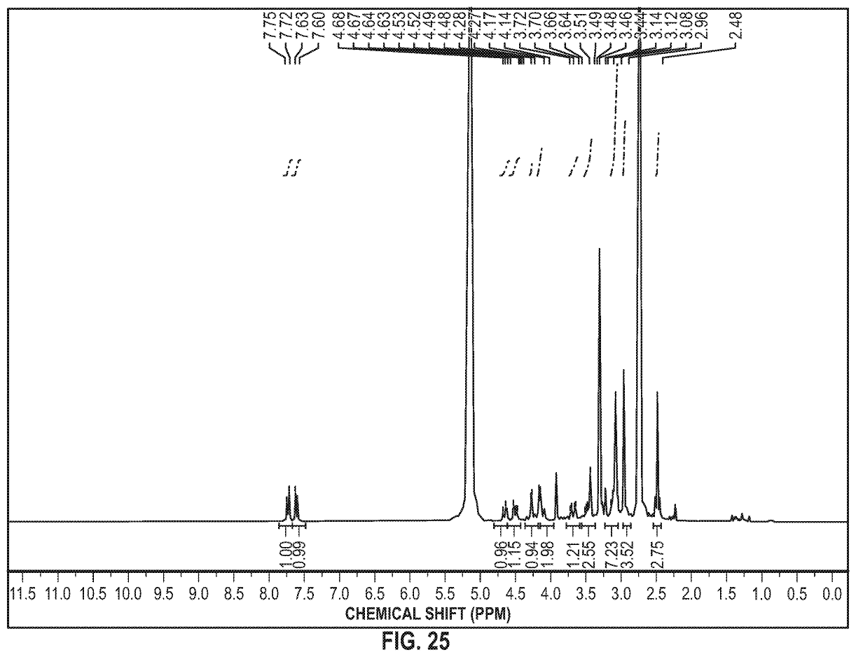

When these same experiments were conducted under blue light irradiation time-dependent inhibition of Tet50 by aTC and compounds 103, 104, and 105 was observed (FIG. 6). aTC was known to be photosensitive and underwent rapid photooxidation which possibly produced a reactive intermediate that inactivated Tet50. Hence, the photosensitivity of aTC derivatives was likely due to the aTC core, and not the side chains. The apparent decay rates under blue light irradiation were significantly faster than the observed rates for reactions that were protected from light. The apparent single-phase exponential decay functions under blue light produced apparent decay constants of 0.6±0.2 min−1, 0.9±0.3 min−1, and 0.5±0.1 min−1 for 103, 104, and 105, respectively, and the apparent half-life values of the decay functions were ~1.2 min, ~0.8 min, and ~1.4 min, respectively. Compound 104 showed slower time-dependent inactivation relative to aTC, compound 103, and compound 105 under these conditions and we were unable to confidently fit the data for compound 104 against Tet50 under blue light to an exponential decay function. Over 7 min, the apparent IC₅₀ values for aTC (106), compound 103, and compound 105 decreased from 500±60 μM to 70±10 μM, from 430±40 μM to 41±5 μM, and from ~1200 μM to 107±6 μM, respectively. A similar light-induced enhancement in the dose-dependent inhibition of Tet50 was observed for the nonspecific probe molecules 101 and 102 (FIG. 18). Collectively, these results suggested that the substitution of aTC at the C9-position with cyclopropylamine (103) or propargylamine (104 and 105) groups imparted time-dependence for the inhibition of Type 2 TDases without the need for photoirradiation.

Analogous time- and light-dependent experiments were performed on PHBH using an NADPH consumption assay. It was important to note that monitoring for NADPH consumption was not a direct indicator of inhibition since uncoupled formation of H₂O₂ possibly provided a false positive and there was no direct monitoring for consumption of the natural substrate para-hydroxybenzoic acid with these inhibitors. In the dark, no inhibition of PHBH was detected for compounds 101 and 102 at concentrations up to 2.5 mM. However, with blue light irradiation potent time-dependent and dose-dependent inhibition of PHBH by 101 and 102 was observed (FIG. 18). Inhibition was detected for aTC, compound 103, and compound 104 under dark conditions. The apparent IC₅₀ values were similar to those observed for Tet50 (Table 1) and time-dependence was observed for compounds 103 and 104, but not aTC (FIG. 19). The apparent PHBH activity decay kinetic were generally slower than those observed for Tet50, but these findings still indicate that propargylamine- and cyclopropylamine-based TDase inhibitors could have had off-target effects on other FAD-containing enzymes.

aTC was known to bind Type 1 TDases in a substrate-like binding mode predicted to orient the D-ring toward the opening of the active site and position a C9-substituent away from the FAD cofactor and into the open solvent space (FIG. 2). It was uncertain whether the aTC derivatives would adopt a binding conformation that facilitates a reaction with the FAD cofactor. Time-dependence of the inhibition of the Type 1 TDase TetX7 under the same conditions described previously for Tet50 was tested. Contrary to Type 2 TDase inhibition, time-dependence for the inhibition of the Type 1 TDase TetX7 by aTC, compound 103, compound 104, or compound 105 was not observed (FIG. 6). It was not possible to explore the time dependence of the effects of the inhibitors under blue light irradiation because of the photoinstability of TetX7 (FIG. 11). These findings were consistent with previous reporting that C10-benzoate ester derivatives of aTC and C9-glycylcycline derivatives of TC bind to Type 1 TDases in a substrate-like orientation, with the bulky D-ring substituent pointing into the open solvent space (FIG. 2).

### Example 16

### Reversibility of TDase Inhibition

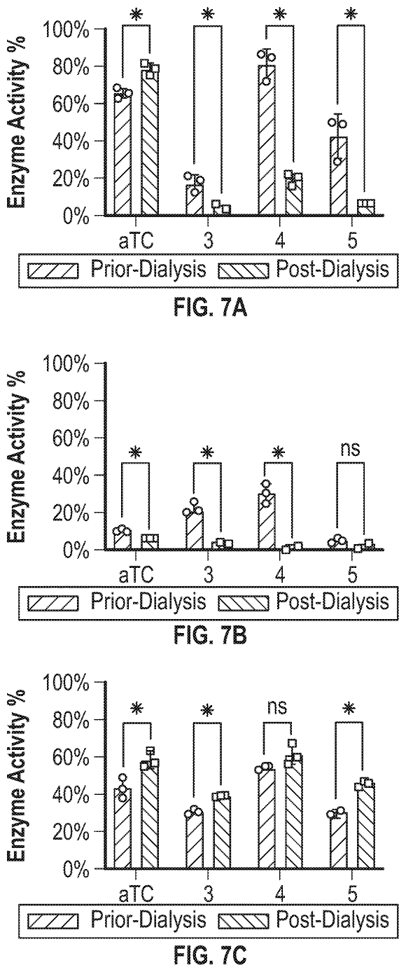

In addition to their time-dependence, covalent enzyme inhibitors resulted in irreversible inhibition. The reversibility of Type 1 (Tet50) and Type 2 (TetX7) TDase inhibition by aTC (106), 103, 104, and 105 was tested via exhaustive dialysis of the respective enzyme-inhibitor complexes. Tet50 was preincubated with aTC, 103, 104, or 105 at concentrations near the apparent IC₅₀ values (Table 1) for 60 min in the dark or 15 min under blue light irradiation followed by exhaustive dialysis for ~24 h at 4 °C. TetX7 was treated under the same conditions but only in the dark because of the photoinstability of TetX7 under blue light irradiation (FIG. 11). TDase activity was measured before and after dialysis relative to that of a no-inhibitor control reaction, which was normalized to 100% activity. For Tet50 under dark conditions, a significant recovery of enzymatic activity after dialysis of the aTC-treated samples was observed, whereas the samples treated with compounds 103, 104, and 105 produced a significant decrease in Tet50 activity after dialysis (FIG. 7). Under blue light irradiation, all three inhibitors including aTC produced greater inhibition of Tet50 postdialysis (FIG. 7). The treatment of TetX7 with all three inhibitors in the dark resulted in a significant recovery of activity after exhaustive dialysis (FIG. 7). Collectively, both exhaustive dialysis and time-dependent inhibition studies supported a model of irreversible inhibition by compounds 103, 104, and 105 against Tet50 under dark conditions and a model of reversible inhibition by aTC and compounds 103, 104, and 105 against TetX7 under dark conditions. The inhibition of Tet50 by aTC under dark conditions was also reversible, whereas blue light irradiation of Tet50 in the presence of aTC and compounds 103, 104, and 105 resulted in time-dependent irreversible inactivation. These results were consistent with the proposed inhibitor binding modes for Type 1 TDase TetX7 (FIG. 2 and FIG. 17) and Type 2 TDase Tet50 (FIG. 4) and supported the observed covalent tagging of FAD only in Type 2 TDases by the cyclopropylamine and propargylamine warheads appended to the C9 position of aTC.

### Example 17

### Covalent TDase Inhibitors Rescue TC Antibacterial Activity

With strong supporting evidence that compounds 103, 104, and 105 acted as selective TDase inhibitors in vitro, these inhibitors were tested in combination with TC against strains of E. coli DH5αZ1 expressing TetX7 (Type 1) or Tet50 (Type 2) from an inducible pZE24 plasmid. Minimum inhibitory concentrations (MICs) were determined using the broth microdilution method according to CLSI guidelines. Visual inspection of bacterial growth and measurement of the OD₆₀₀ were used to determine the lowest concentration at which bacterial growth was inhibited. The MICs for TC against the empty vector control, TetX7::pZE24, and Tet50::pZE24 were 1, 16, and 16 μg/mL, respectively. In prior work, it was reported the MICs of aTC against the empty vector control, TetX7::pZE24, and Tet50::pZE24 as 4, 8, and 8 μg/mL, respectively, and it was found that aTC at as low as 2 μg/mL rescued the growth inhibitory activity of TC at 8 μg/mL against resistant strains. The MIC values for compounds 103, 104, and 105 were determined against the empty vector control, TetX7::pZE24, and Tet50::pZE24 strains of E. coli DH5αZ1 in the absence of TC and in the presence of TC at 8 μg/mL. Compounds 103, 104, and 105 displayed moderate growth inhibitory activity with MICs ranging from 16-64 μg/mL against the three strains (Table 2). The addition of 8 μg/mL TC significantly increased the growth inhibitory activity of the TC/inhibitor combinations against all strains. The apparent MICs of the inhibitors against resistant strains were 4-to-8-fold lower than those of the inhibitor or TC alone. While cyclopropylamine 103 was generally more potent than propargylamines 104 and 105 against TetX7 and Tet50 in vitro (Table 1), propargylamine 104 was the best TDase inhibitor in live cells. Compound 104 had optimal activity against TetX7 with an observed recovery of TC growth inhibition at 2 μg/mL, which was in line with the best TDase inhibitors developed to date.

TABLE 2
Minimum Inhibitory Concentration (MIC in μg/mL) against
E. coli DH5αZ1 Expressing TetX7 or Tet50 from the pZE24
Plasmid in the Absence (−TC) or Presence (+TC) of 8 μg/mL TC

| | | | |
|---|---|---|---|
| Strain/ | Empty | TetX7 | Tet50 |

| compound | −TC | +TC | −TC | +TC | −TC | +TC |
|---|---|---|---|---|---|---|
| 103 | 32 | <1 | 64 | 8 | 64 | 16 |
| 104 | 32 | <1 | 16 | 2 | 32 | 8 |
| 105 | 63 | <1 | 64 | 2 | 32 | 16 |

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 3
List of Reference Characters

| | |
|---|---|
| 101 | N-(1-methyl)cyclopropylbenzylamine |
| 102 | N-methyl-N-benzyl-propargylamine |
| 103 | Reaction product of anhydrotetracycline with cyclopropylamine |
| 104 | Reaction product of anhydrotetracycline with propargylamine |
| 105 | Reaction product of anhydrotetracycline with methyl-substituted |
| | propargylamine |
| 106 | Anhydrotetracycline |
| 107 | Flavin adenine dinucleotide |

The disclosure being thus described, it will be obvious that the same may be varied in many ways. Such variations are not to be regarded as a departure from the spirit and scope of the disclosures and all such modifications are intended to be included within the scope of the following claims. The above specification provides a description of the manufacture and use of the disclosed compositions and methods. Since many embodiments can be made without departing from the spirit and scope of the disclosure, the invention resides in the claims.

What is claimed is:

1. A composition for treating tetracycline resistance comprising:
a tetracycline compound, wherein the tetracycline compound comprises tetracycline, a tetracycline derivative, a tetracycline analog, a pharmaceutically acceptable salt of the foregoing, and/or mixtures thereof;
wherein the tetracycline compound has a warhead capable of forming a covalent bond with a flavin adenine dinucleotide.

2. The composition of claim 1, wherein the warhead comprises a cyclopropylamine and/or a propargylamine.

3. The composition of claim 1, wherein the tetracycline compound comprises an anhydrotetracycline, a chlortetracycline, an oxytetracycline, and/or a demeclocycline.

4. The composition of claim 3, wherein the tetracycline compound comprises a derivative of anhydrotetracycline and/or pharmaceutically acceptable salt thereof.

5. The composition of claim 4, wherein the derivative of anhydrotetracycline comprises a modification at the C9 position.

6. The composition of claim 1, wherein the tetracycline compound inhibits a flavoenzyme.

7. The composition of claim 6, wherein the flavoenzyme comprises a tetracycline destructase.

8. The composition of claim 7, wherein the tetracycline destructase is a Type I or Type II tetracycline destructase selected from the group consisting of Tet47, Tet48, Tet49, Tet50, Tet51, Tet52, Tet54, Tet55, Tet56, TetX, and combinations thereof.

9. The composition of claim 1, further comprising a tetracycline antibiotic.

10. The composition of claim 1, further comprising a pharmaceutically acceptable excipient.

11. A method of treating a bacterial infection in a subject, the method comprising: administering a therapeutically effective amount of the composition of claim 1.

12. The method of claim 11, wherein the tetracycline compound comprises an anhydrotetracycline, a chlortetracycline, an oxytetracycline, and/or a demeclocycline; and wherein the warhead comprises a cyclopropylamine and/or a propargylamine.

13. The method of claim 12, wherein the tetracycline comprises a derivative of anhydrotetracycline; and wherein the derivative of anhydrotetracycline comprises a modification at the C9 position.

14. The method of claim 11, wherein the tetracycline forms a covalent adduct with a flavin adenine dinucleotide.

15. The method of claim 14, wherein the covalent adduct binds to and/or inhibits a flavoenzyme.

16. The method of claim 15, wherein the flavoenzyme is encoded by the bacteria.

17. The method of claim 16, wherein the flavoenzyme is a Type I or Type II tetracycline destructase selected from the group consisting of Tet47, Tet48, Tet49, Tet50, Tet51, Tet52, Tet54, Tet55, Tet56, TetX, and combinations thereof.

18. The method of claim 17, further comprising administering to the subject a tetracycline antibiotic.

19. The method of claim 18, wherein the tetracycline antibiotic exhibits antibiotic activity against the bacteria.

20. The method of claim 11, wherein the composition further comprises a pharmaceutically acceptable excipient.
